(19) United States
(12) Patent Application Publication
(10) Pub. No.: US 2010/0191216 A1
(43) Pub. Date: Jul. 29, 2010

(54) INDICATIONS FOR LOCAL TRANSPORT OF ANAESTHETIC AGENTS BY ELECTROTRANSPORT DEVICES

(76) Inventors: Preston Keusch, Jonesboro, ME (US); Vilambi Nrk Reddy, Tamll Nadu (IN); Richard Greene, Walterboro, SC (US); George M. Baskinger, North Haledon, NJ (US); Ashutosh Sharma, Springfield, NJ (US)

Correspondence Address: K&L GATES LLP, 210 SIXTH AVENUE, PITTSBURGH, PA, 15222-2613, US

(21) Appl. No.: 12/492,361

(22) Filed: Jun. 26, 2009

Related U.S. Application Data
(63) Continuation of application No. 11/537,182, filed on Sep. 29, 2006, now abandoned.
(60) Provisional application No. 60/722,603, filed on Sep. 30, 2005.

(51) Int. Cl. A61N 1/30

(52) U.S. Cl. 604/501

(57) ABSTRACT

The use of an iontophoresis electrode assembly for delivery of a drug formulation is described. The drug formulation includes an anaesthetic and a vasoconstrictor. It is administered to a patient prior to a procedure to produce clinically acceptable depth and duration of dermal anaesthesia at the portion of skin to subject to a painful procedure or to reduce or eliminate pain. The procedure is one selected from the group consisting of venipuncture, IV cannulation, needle aspirations, body piercings, blood donations, electrolysis, tattoo removal, tattoo application, injections, dermabrasion, skin peeling, high velocity particle ablation, pace maker implantation, pace maker replacement, epidural puncture, lumbar puncture, regional nerve blocks, skin harvesting, small skin incisions, skin biopsies, circumcisions or excisions. The iontophoresis electrode assembly may also be used to reduce or temporarily eliminate neuropathic pain.

### CROSS REFERENCE TO RELATED APPLICATION

This application claims priority from U.S. provisional patent application Ser. No. 60/722,603.

### STATEMENT REGARDING FEDERALLY SPONSORED RESEARCH

Not Applicable

### BACKGROUND

### Description of the Related Art

The present invention relates to various indications for use of electrotransport devices for the local delivery of analgesics and other drugs. Transdermal drug delivery systems have, in recent years, become an increasingly important means of administering drugs. Such systems offer advantages clearly not achievable by other modes of administration such as introduction of the drug through the gastro-intestinal tract or punctures in the skin, to name a few.

There are two types of transdermal drug delivery systems, “passive” and “active.” Passive systems deliver drug through the skin of the user unaided, an example of which would involve the application of a topical anaesthetic to provide localized relief, as disclosed in U.S. Pat. No. 3,814,095. Active systems, on the other hand, use external force to facilitate delivery of a drug through a patient's skin. Examples of active systems include electrotransport, ultrasound, electroporation and/or iontophoresis.

Iontophoretic drug delivery is the migration of drug ions through the skin in response to the establishment of an electrical potential. By passing a weak electrical current through a suitably designed transdermal drug delivery patch, a drug ion of a particular charge contained in a specially designed reservoir may be driven out of the reservoir and into intact skin. Iontophoretic delivery of a medicament is accomplished by application of a voltage to a medicament-loaded reservoir-electrode, sufficient to maintain a current between the medicament-loaded reservoir-electrode and a return reservoir electrode (another electrode) applied to a patient's skin so that the desired medicament is delivered to the patient in ionic form.

Conventional iontophoretic devices, such as those described in U.S. Pat. Nos. 4,820,263, 4,927,408, and 5,084,008, deliver a drug transdermally by iontophoresis. These devices basically consist of two electrodes—an anode and a cathode. In a typical iontophoretic device, electric current is driven from an external power supply. In a device for delivering a drug from an anode, the positively charged drug is delivered into the skin at the anode, with the cathode completing the electrical circuit. Likewise, in a system for delivering a drug from a cathode, the negatively charged drug is delivered into the skin at the cathode, with the anode completing the electrical circuit. Accordingly, there has been considerable interest in iontophoresis to perform delivery of drugs for a variety of purposes. One example is the delivery of lidocaine, a common topical, local anaesthetic.

A further problem related to production of a successful pharmaceutical product is related to the requirements for accuracy and precision of dosage. In some of the iontophoretic drug delivery devices described above, the user or the practitioner is required to perform some action to hydrate the reservoir-electrode and introduce the medicament to be delivered into the delivery device prior to use. Such operations that depend upon the practitioner or user to charge the medicament into the device under relatively uncontrolled conditions may result in improper dosing. Regulatory requirements for pharmaceutical products generally specify not only that medicaments contain between ninety and one hundred-ten percent of the label claim, but also that the delivery be uniform from sample to sample. It is well recognized that many medicaments are not stable under conditions necessary for assembly and storage of iontophoretic reservoir-electrodes. A method of accurately and repeatedly loading the medicament and any required stability enhancing excipients during the assembly process of reservoirs useful for passive transdermal drug delivery and reservoir-electrodes for iontophoretic drug delivery devices, that is compatible with a mechanized assembly process and also provides a drug charged reservoir-electrode with satisfactory stability properties is described in U.S. Pat. No. 6,496,727, which is incorporated herein by reference in its entirety.

Iontophoresis devices for delivery of lidocaine heretofore available fail to provide sufficient stability for extended shelf life.

Stability of a commercially acceptable iontophoretic system for delivery of lidocaine and epinephrine involves considerations well beyond drug stability as compared to storing an aqueous lidocaine/epinephrine anaesthetic solution packaged in glass vials or even in a pre-filled syringe.

### BRIEF SUMMARY OF THE INVENTION

An integrated electrode assembly structured for use in an electrically assisted delivery device for delivery of a composition, such as a drug formulation, through a membrane is provided in co-pending application Ser. No. 10/820,346 filed Apr. 7, 2004, which is incorporated herein by reference in its entirety. One embodiment of that assembly is composed of a drug-filled patch connected to a source of electrical current (controller). Both the anode and cathode assemblies reside in a single patch. The patch anode contains the drug formulation, while the cathode acts as a return electrode during active treatment. The drug formulation contained in the anode is comprised of an anaesthetic. In embodiments where delivery is limited to the dermal layers, a vasoconstrictor, which lengthens the duration of the anaesthetic response and limits the systemic uptake of the anaesthesia, may be added. The anaesthetic may be, for example, lidocaine HCl, and the vasoconstrictor may be, for example, epinephrine or phenylephrine. The controller is an electronic system (including hardware and interconnect) designed to provide a pre-programmed direct current for transdermal iontophoretic drug delivery.

In various embodiments, the integrated electrode assembly includes a flexible backing; an electrode layer connected to the flexible backing, the electrode layer having at least a donor electrode and a return electrode; at least one lead extending from each of the donor electrode and the return electrode to a tab end portion of the assembly, the tab end portion being structured for electrical connection with a source of electrical current; a donor reservoir positioned in communication with the donor electrode, the donor reservoir including an amount of a drug formulation; and, a return reservoir positioned in communication with the return electrode.

An alternative embodiment of the electrode assembly includes a split patch design having separate anode and cathode portions.

The improvement in the depth and duration of the anaesthetic response provided by the integrated electrode assembly described above opens the use of electrical assisted delivery of local anaesthetics for a wide variety of dermal and epidermal treatments for which anaesthetic injection was the accepted means of delivering local anaesthesia. Examples of indications for which electrically assisted delivery of local anaesthesia is now preferred include venipuncture, IV cannulation, incision and excision, and laser treatment of the dermal layers and skin surface removal procedures.

Use of the iontophoretic delivery of local anaesthesia to ease the pain and emotional trauma of venipuncture, IV cannulation, and injections for children (defined as birth up to 18 years of age) is of particular importance. Similar puncture type procedures, such as needle aspirations, body piercings, blood donations, injections, tattoo applications, epidural punctures, lumbar punctures and regional nerve blocks are also suitable indications for iontophoretic delivery of anaesthesia.

Other indications include incision and excision procedures, such as the removal of skin lesions, biopsies, circumcisions, subcutaneous implantation of drug depots, removal of pacemakers, subcutaneous implantation of replacement pacemakers, removal of scar tissue and skin harvesting. Skin lesions which may be removed following the electrically assisted delivery of an anaesthetic and vasoconstrictor include, for example, actinic keratosis, angioma, hemangioma, basal cell epithelioma, Clarks nevus, cysts, dermafibroma, hyperkeratotic lesions, moles, sebhorrheic keratosis, skin tags, skin nodules, squamous cell carcinoma and warts.

Other indications include laser procedures, such as the laser removal of any of the aforementioned skin lesions, removal of tattoos, removal of scar tissue, laser resurfacing of skin and dermabrasion. Skin surface removal procedures include, for example, electrolysis, tattoo removal, dermabrasion, skin peeling, high velocity particle ablation and skin harvesting.

In an embodiment wherein a vasoconstrictor is not added so that the anaesthetic has systemic affect, the electrically assisted delivery of anaesthetic may be used to treat chronic refractory pain resulting from any cause including neuropathic pain, cancer, diabetic neuropathy, neuropathy of shingles, postherpetic neuralgia and trigeminal neuralgia. The amount of lidocaine delivered systemically will be kept well below blood levels associated with central nervous system or cardiovascular toxicity.

Embodiments of the integrated electrode assembly may include at least one of the following features and combinations thereof: an insulating dielectric coating positioned adjacent to at least a portion of at least one of the electrodes and the leads; at least one spline formed in the electrode layer; a tab stiffener connected to the tab end portion; a tab slit formed in the tab end portion; a sensor trace positioned on the tab end portion; a release cover having a donor portion structured to cover the donor reservoir and a return portion structured to cover the return reservoir; at least a portion of the flexible backing having a flexural rigidity less than a flexural rigidity of at least a portion of the electrode layer; a shortest distance between a surface area of an assembly including the donor electrode and the donor reservoir and a surface area of an assembly including the return electrode and the return reservoir being sized to provide a substantially uniform path of delivery for the composition through the membrane; a surface area of an assembly including the donor electrode and the donor reservoir is greater than a surface area of an assembly including the return electrode and the return reservoir; a ratio of a surface area of at least one of the reservoirs to a surface area of its corresponding electrode is in the range of about 1.0 to 1.5; a footprint area of the assembly is in the range of about 3 cm² to 100 cm², more preferably in the range of about 5 cm² to 60 cm², and most preferably in the range of about 20 cm² to 30 cm²; a ratio of a total surface area of the electrodes to a total footprint area of the assembly is in the range of about 0.1 to 0.7; a ratio of a surface area of the donor electrode to a surface area of the return electrode is in the range of about 0.1 to 5.0; a ratio of a thickness of the donor reservoir to a thickness of the return reservoir is in the range of about 0.2 to 3.0; at least one component of the assembly in communication with at least one of the reservoirs has an aqueous absorption capacity less than an aqueous absorption capacity of the reservoir in communication with the component of the assembly; a slit formed in the flexible backing in an area located between the donor electrode and the return electrode; at least one non-adhesive tab extending from the flexible backing; a gap formed between a portion of a layer of transfer adhesive deposited on the electrode layer and a portion of a tab stiffener connected to the tab end portion; a tab stiffener attached to a portion of the tab end portion; at least one tactile sensation aid formed in the tab end portion; at least one indicium formed on at least a portion of the assembly; a minimum width of a portion of a layer of transfer adhesive deposited on the electrode layer adjacent to at least one of the donor electrode and the return electrode is in the range of at least about 0.9 cm; or, a minimum tab length associated with the tab end portion is in the range of at least about 3.5 cm.

The use of the integrated electrode assembly for the electrically assisted delivery of anaesthetic, alone or in combination with a vasoconstrictor, to alleviate the discomfort of medical procedures and/or pain due to disease is described in more detail herein below.

### BRIEF DESCRIPTION OF THE DRAWINGS

FIG. 1 shows an exploded isometric view of various aspects of the Platform I embodiment of an integrated electrode assembly.

FIG. 2 shows an exploded isometric view of various aspects of an integrated electrode assembly of FIG. 1.

FIG. 3 shows an elevated view of various aspects of an integrated electrode of FIG. 2.

FIG. 4A includes an exploded isometric view illustrating various aspects of the interconnection of an integrated electrode assembly with components of an electrically assisted delivery device.

FIG. 4B shows a schematic representation of the interaction between a portion of an integrated electrode assembly and components of an electrically assisted delivery device.

FIG. 4C illustrates a schematic representation of the interaction between a portion of an integrated electrode assembly and components of an electrically assisted delivery device.

FIG. 5A includes a schematic elevated view of various aspects of an integrated electrode assembly.

FIGS. 5B and 5C show cross-sectional views illustrating aspects of the electrode assembly of FIG. 5A.

FIG. 6 includes a schematic elevated view of various aspects of an integrated electrode assembly.

FIG. 7 includes a cross-sectional view of the release cover of FIG. 6.

FIG. 8 includes a schematic that illustrates the effect of electrode geometry and spacing on the delivery paths of a composition through a membrane.

FIG. 9 includes a schematic that illustrates the effect of electrode geometry and spacing on the delivery paths of a composition through a membrane.

FIG. 10 shows a cross-sectional view of a schematic un-loaded electrode assembly in contact with a loading solution.

FIG. 11 is a cut-away view of a package including one embodiment of an electrode assembly described herein.

FIG. 12 is a view of the Platform IIA iontophoretic integrated electrode assembly.

FIG. 13 is a view of the Platform IIB iontophoretic integrated electrode assembly.

FIG. 14 is a view of the Platform III iontophoretic split patch electrode assembly.

FIG. 15 illustrates the Nine-Face Interval Scale used to evaluate the occurrence and extent of pain experienced by children who were involved in one or more studies described herein.

### DETAILED DESCRIPTION

The present invention is directed to the various uses to which electrically assisted delivery of an anaesthetic is indicated. Experiments done to test the effectiveness of various categories of such indications are described herein below. The tests were done with one or more of four main types of electrically assisted delivery platforms, designated Platform I, IIA, IIB and III herein. Each Platform will be described more fully below.

### DEFINITIONS

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. Also, the disclosure of these ranges is intended as a continuous range including every value between the minimum and maximum values.

Unless otherwise specified, embodiments of the present invention are employed under “normal use” conditions, which refer to use within standard operating parameters for those embodiments. During operation of various embodiments described herein, a deviation from a target value of one or more parameters of about ±10% or less for an iontophoretic device under “normal use” is considered an adequate deviation for purposes of the present invention.

As used herein, “anaesthesia” refers to a state characterized by a loss of sensation as a result of pharmacologic depression of nerve function. As used herein, the terms “anaesthetic” refers to a compound or drug formulation that produces a loss of sensation as a result of depression of nerve function. “Anaesthesia” and “anaesthetic” are synonymous with “analgesia” and “analgesics” in that a patient's state of consciousness is not considered when referring to local effects of use of the described iontophoretic device, even though some of the drugs mentioned herein below may be better classified as “analgesics” or “anaesthetics” in their systemic use.

As used herein, “non-necrotizing” refers to not causing necrosis, wherein necrosis is defined as death of tissues or cells caused when not enough blood is supplied to the tissues or cells due to injury. With particular reference to “non-necrotizing amount of vasoconstrictor,” the amount of vasoconstrictor delivered in the invention does not cause the tissue in contact with the vasoconstrictor to be injured to the point wherein blood supply is substantially compromised, causing cellular death.

The terms “unloaded” or “unloaded reservoir,” are necessarily defined by the process of loading a reservoir. In the loading process, a drug or other compound or composition is absorbed, adsorbed and/or diffused into a reservoir to reach a final content or concentration of the compound or composition. An unloaded reservoir is a reservoir that lacks that compound or composition in its final content or concentration. In one example, the unloaded drug reservoir is a hydrogel, as described in further detail below that includes water and a salt. Although the salt may be one of many salts, including alkaline metal halide salts, the salt typically is sodium chloride. Other halide salts such as, without limitation, KCl or LiCl might be equal to NaCl in terms of functionality, but may not be preferred. Use of halide salts to prevent electrode corrosion is disclosed in U.S. Pat. Nos. 6,629,968 and 6,635,045. One or more additional ingredients may be included in the unloaded reservoir. Typically, active ingredients are not present in the unloaded gel reservoir. Other additional, typically non-ionic ingredients, such as preservatives, may be included in the unloaded reservoir.

The term “electrically assisted delivery” refers to the facilitation of the transfer of any compound across a membrane, such as, without limitation, skin, mucous membranes and nails, by the application of an electric potential across that membrane. “Electrically assisted delivery” is intended to include, without limitation, iontophoretic, electrophoretic and electroendosmotic delivery methods.

By “active ingredient,” it is meant, without limitation, drugs, active agents, therapeutic compounds and any other compound capable of eliciting any pharmacological effect in the recipient that is capable of transfer by electrically assisted delivery methods. A “transdermal device” or “transdermal patch” includes both active and passive transdermal devices or patches.

As applied to various embodiments of electrically assisted delivery devices described herein, the term “integrated” as used in connection with a device indicates that at least two electrodes are associated with a common structural element of the device. For example, and without limitation, a transdermal patch of an iontophoretic device may include both a cathode and an anode “integrated” therein, e.g., the cathode and anode are attached to a common backing.

As applied to various embodiments of electrically assisted delivery devices described herein, a “flexible” material or structural component is generally compliant and conformable to a variety of membrane surface area configurations and a “stiff” material or structural component is generally not compliant and not conformable to a variety of membrane surface area configurations. In addition, a “flexible” material or component possesses a lower flexural rigidity in comparison to a “stiff” material or structural component having a higher flexural rigidity. For example and without limitation, a flexible material when used as a backing for an integrated patch can substantially conform over the shape of a patient's forearm or inside elbow, whereas a comparatively “stiff” material would not substantially conform in the same use as a backing.

As applied herein, the term “transfer absorbent” includes any media structured to retain therein a fluid or fluids on an at least temporary basis and to release the retained fluids to another medium such as a hydrogel reservoir. Examples of “transfer absorbents” that may be employed herein include, without limitation, non-woven fabrics and open-cell sponges.

The term “lidocaine”, unless otherwise specified, refers to any water-soluble form of lidocaine, including salts or derivatives, homologs or analogs thereof. For example, as is used in Examples below, “lidocaine” refers to lidocaine hydrochloride (HCl), in substantially ionic form, commercially available, for example, as XYLOCAINE® (a trademark of AstraZeneca LP of Wayne, Pa.), among other names.

Lidocaine is a local anaesthetic of the amide type. Lidocaine Hydrochloride, chemically designated as: 2-(Diethylamino)-2′,6′-acetoxylidide mono-hydrochloride, monohydrate, is a white crystalline powder freely soluble in water, with a molecular weight of 288.81.

The molecular formula for 2-(Diethylamino)-2′,6′-acetoxylidide mono-hydrochloride, monohydrate is C₁₄H₂₂N₂O.HCl and its structural formula is:

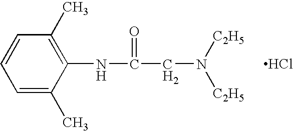

The term “epinephrine” refers to any form of epinephrine, the salts, its free base or derivatives and homologs or analogs thereof so long as they can be solubilized in an aqueous solution. For example, as is used in the examples below, “epinephrine” refers to epinephrine bitartrate.

Epinephrine, a sympathomimetic (adrenergic) agent designated chemically as 1,2 Benzenediol, 4-[1-hydroxy-2-(methylamino)ethyl]-,(R)—, [R—(R*,R*)]-2,3-dihydroxybutanedioate (1:1) (salt), is a white, crystalline powder with a molecular weight of 333.29. Its molecular formula is C₉H₁₃NO₃.C₄H₆O₆ and its structural formula is:

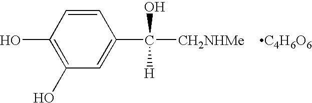

As used herein, “stable” and “stability” refer to a property of individual packaged electrode-reservoir assemblies, and typically is demonstrated statistically. The term “stable” refers to retention of a desired quality of a variety of parameters, with particular, but not exclusive focus on active ingredients such as epinephrine content, lidocaine content, hydrogel strength, hydrogel tack, electrical circuitry and electrical capacity, within a desired range. Drug or pharmaceutical stability is another parameter. For instance, epinephrine typically is very unstable. Therefore, an iontophoretic electrode assembly might be considered stable for the time period that useful quantities of epinephrine remain available for delivery. Similarly, if lidocaine is considered, the electrode assembly remains stable for the time period that useful quantities of lidocaine remain available for delivery.

In an iontophoretic device, the U.S. Food and Drug Administration (FDA) may require retention, as a lot, of 90% of the label claim of epinephrine over a given time period using a least square linear regression statistical method with a 95% confidence level. However, as used herein, an electrode assembly and/or parts thereof, are considered stable so long as they substantially retain their desired function in an iontophoretic system. Stability, though measured by any applicable statistical method, is a quality of the electrode assembly. Therefore, methods other than FDA-approved statistical methods may be used to quantitate stability. For instance, even though for FDA purposes, a 95% confidence level may be required, those limits are not literally required for a device to be called “stable.” Similarly, and for exemplary purposes only, a “stable” iontophoretic electrode may be said to retain 80% of the original epinephrine concentration over a given time period, as determined by least square linear regression analysis.

As used generally herein, an electrode-reservoir, reservoir or electrode assembly is stable when hermetically sealed for a given time period. This means that when the electrode assembly is sealed in a container that is impermeable to oxygen and water (“hermetically sealed”), the electrode-reservoir retains a specified characteristic or parameter within desired boundaries for a given time period. By “original concentration”, “original amounts” or “original levels” it is meant the concentration, amount or level of any constituent or physical, electrochemical or electrical parameter relating to the electrode assembly at a time point designated as t=0, and typically refers to a time point after the electrode assembly is sealed within the hermetically sealed container. This time may take up to a few weeks to ensure uniform distribution of ingredients in the reservoir(s).

### Physical Features of Embodiments of the Electrically Assisted Delivery Devices

One embodiment of an electrically assisted delivery device, referred to as Platform I (100), is a flexible integrated electrode assembly, shown in FIGS. 2-4 and 5A-7. In Platform I, described more fully below, the drug formulation and electrolyte solution are transferred to the reservoirs with absorbent pads.

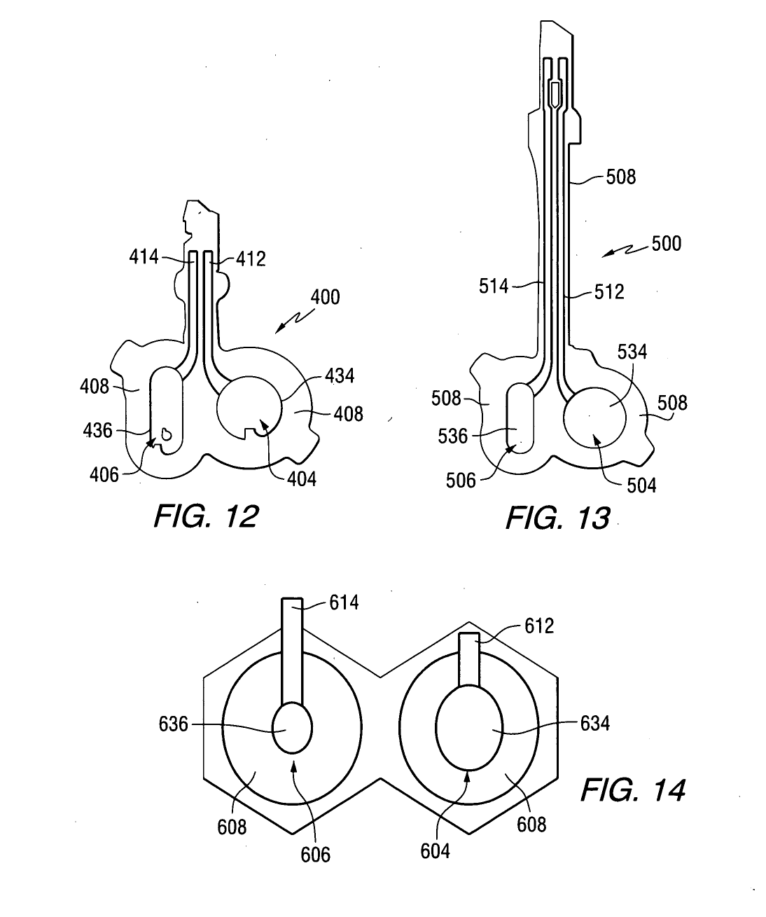

Platform IIA (400), as shown in FIG. 12, has a flexible integrated anode 404 and cathode 406 design in a single patch with electrodes 412, 414 leading to a controller (not shown) identical to Platform I, except that the drug formulation is drop loaded into the drug reservoir 434, 436 of the device 400. The backing 408 in Platform IIA that is in contact with the patient's skin is a flexible material, such as ethylvinylacetate (EVA). The anode and cathode hydrogel reservoirs 434, 436, which may be made of a polyvinylpyrrolidone (PVP) material, contain a salt, for example, NaCl, at about 0.06%, to prevent electrode corrosion during loading of active electrolyte solutions into the reservoirs 434, 436.

Platform IIB (500), shown in FIG. 13, is another embodiment of an integrated electrically assisted delivery device with a side-by-side anode 504 and cathode 506 pattern and longer interconnect traces 512, 514 than in Platforms I and IIA. The backing 508 in Platform IIB (500) is also made of a flexible material, such as EVA, with polyethylene terephthalate (“PET”) limited to the back of the silver/silver chloride electrodes and traces 512, 514. A dielectric coating is preferably placed on the traces and around the periphery of the electrodes to limit the possibility of the electrode touching the skin. Aqueous solutions of the active ingredients of the drug formulation were loaded onto the anode 504 and aqueous solutions of the electrolyte were loaded onto the cathode 506 by placement of the said solutions onto the PVP hydrogel reservoirs 534, 536. The PVP hydrogel reservoirs 534, 536 consist of about 16% by weight of cross-linked PVP adhered to silver/silver chloride printed electrodes.

Platform III is a split patch design 600, shown in FIG. 14. There are separate anode 604 and cathode 606 portions with the electrodes 612, 614 connected by wires to a controller (not shown). The patient side of the anode 604 contains the drug formulation and the return cathode 606 contains an electrolyte. Aqueous solutions of the active ingredients of the drug formulation and electrolyte were loaded in the same way as in Platforms II, onto the anode and cathode surfaces, respectively; in a PVP hydrogel reservoir 634, 636 consisting of about 16% by weight of cross-linked PVP adhered to silver/silver chloride printed electrodes 612, 614. In one embodiment, the anode is about 5 cm² and the cathode portion is about 3-4 cm². A peripheral adhesive, made for example of an acrylic material, surrounds each patch.

Platforms I, IIA, IIB and III are sometimes referred to herein as the “Electrotransport device” and when used together with the drug to be delivered, the “Electrotransport System”. Because each of these platforms are electrically and chemically the same, they are functionally equivalent regarding their electrotransport activity.

The following description of Platform I is found in co-pending application Ser. No. 10/820,346 filed Apr. 7, 2004, incorporated herein by reference. Referring to FIGS. 2-4, a printed electrode layer 102, including two electrodes (an anode 104 and a cathode 106), is connected to a flexible backing 108 by a layer of flexible transfer adhesive 110 positioned between the printed electrode layer 102 and the flexible backing 108. One or more leads 112, 114 may extend from the anode 104 and/or cathode 106 to a tab end portion 116 of the printed electrode layer 102. In various aspects, an insulating dielectric coating may be deposited on and/or adjacent to at least a portion of one or more of the electrodes 104, 106 and/or the leads 112, 114. The dielectric coating may serve to strengthen or bolster the physical integrity of the printed electrode layer 102; to reduce point source concentrations of current passing through the leads 112, 114 and/or the electrodes 104, 106; and/or to resist creating an undesired short circuit path between portions of the anode 104 and its associated lead 112 and portions of the cathode 106 and its associated lead 114.

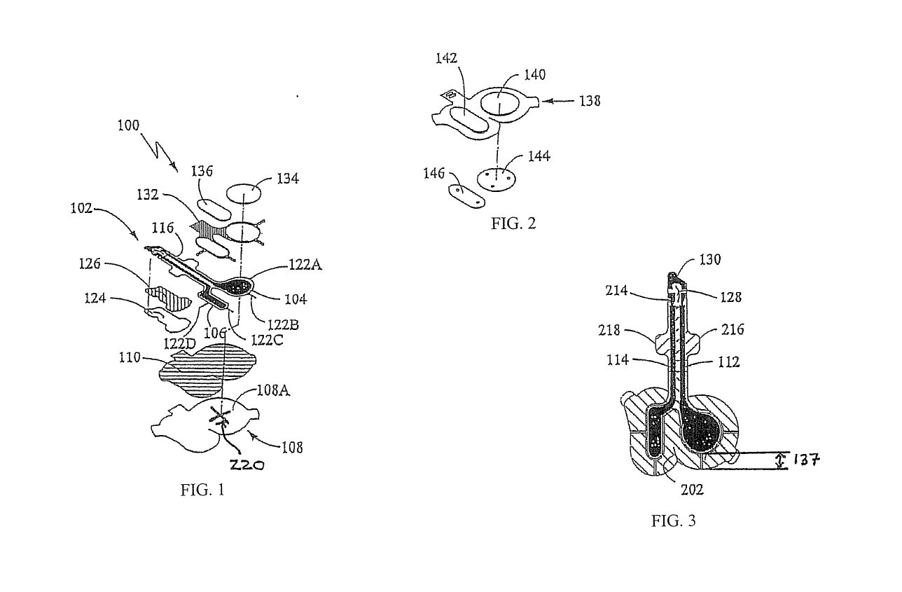

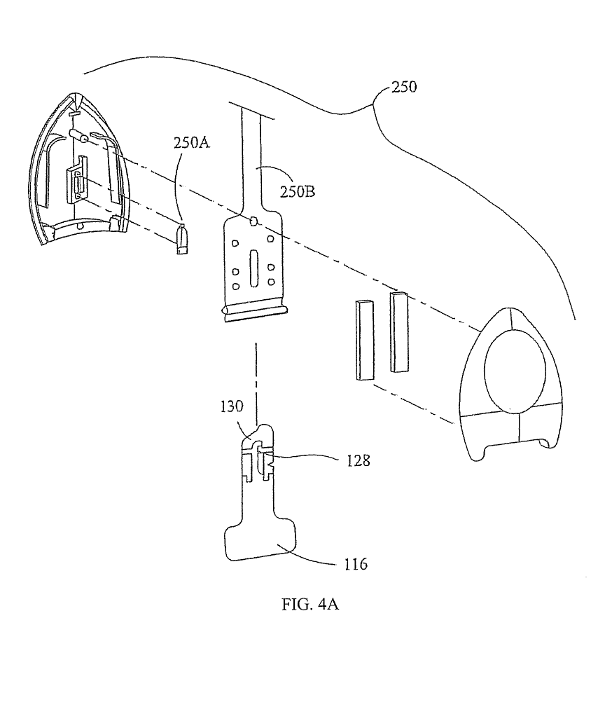

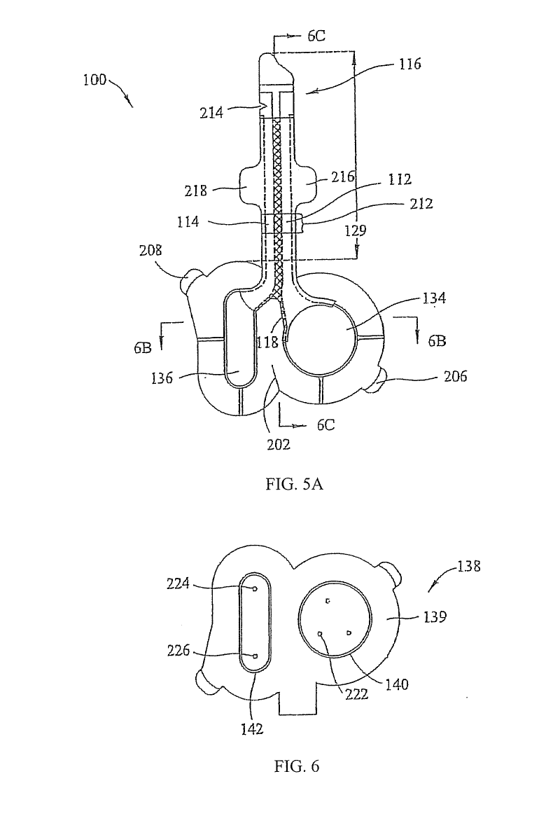

In certain non-limiting embodiments of the present invention, a tab stiffener 124 is connected to the tab end portion 116 of the printed electrode layer 102 by a layer of adhesive 126 positioned between the tab stiffener 124 and the tab end portion 116. In various embodiments, a tab slit 128 may be formed in the tab end portion 116 of the assembly 100 (as shown more particularly in FIGS. 1 and 3). The tab slit 128 may be formed to extend through the tab stiffener 124 and the layer of adhesive 126. In other embodiments, a minimum tab length 129 (as shown particularly in FIG. 5A) for the depicted embodiment as structured in association with the tab end portion 116 may be in the range of at least about 3.5 cm.

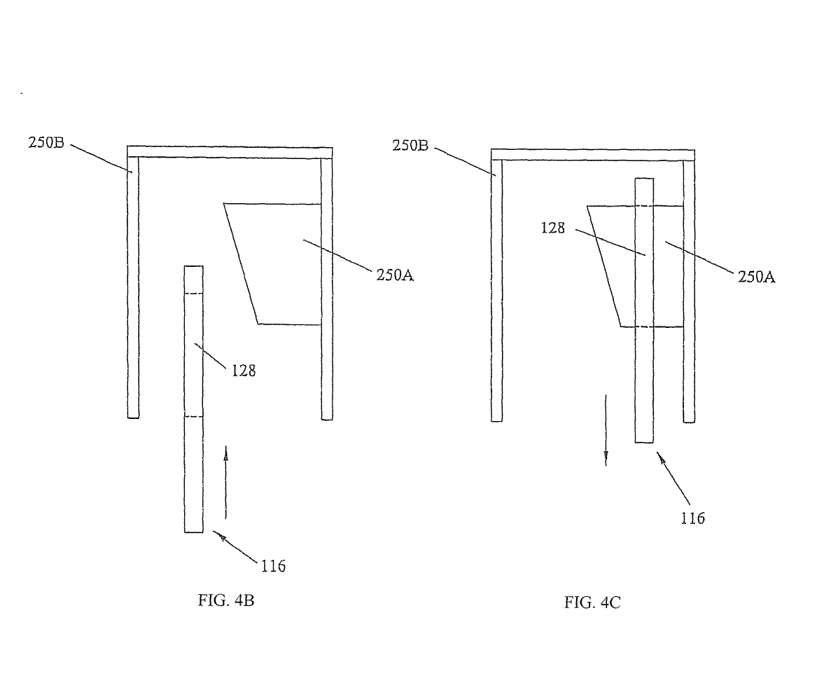

With reference to FIGS. 4A-4C, the tab end portion 116 may be structured to be mechanically or electrically operatively associated with one or more other components of an electrically assisted drug delivery device such as a knife edge 250A of a connector assembly 250, for example. As shown schematically in FIGS. 4B and 4C, once the tab end portion 116 is inserted into a flexible circuit connector 250B of the connector assembly 250, the tab slit 128 of the tab end portion 116 may be structured to receive therein the knife edge 250A. It can be appreciated that the interaction between the knife edge 250A and the tab slit 128 may serve as a tactile sensation aid for a user manually inserting the tab end portion 116 into the flexible circuit connector 250B of the connector assembly 250. In addition, the knife edge 250A may be structured, upon removal of the tab end portion 116 from the connector assembly 250, to cut or otherwise disable one or more electrical contact portions positioned on the tab end portion 116, such as a sensor trace 130, for example. It can be seen that this disablement of the electrical contact portions may reduce the likelihood that unintended future uses of the assembly 100 will occur after an initial use of the assembly 100 and the connector assembly 250 for delivery of a composition to a membrane, for example.

In other aspects, a layer of transfer adhesive 110 may be positioned in communication with the printed electrode layer 102 to facilitate adherence and/or removal of the assembly 100 from a membrane; for example, during operation of an electrically assisted delivery device that includes the assembly 100. As shown in FIG. 1, a first hydrogel reservoir 134 is positioned for communication with the anode 104 of the printed electrode layer 102 and a second hydrogel reservoir 136 is positioned for communication with the cathode 106 of the printed electrode layer 102. In other aspects, although a hydrogel may be preferred in many instances, there may be substantially no hydrogel reservoir associated with the cathode 106, or a substance including NaCl, for example, may be associated with the cathode 106.

As shown in FIG. 2, a release cover 138 includes an anode-donor portion 140 and a cathode-return portion 142. The anode-donor portion 140 is structured to receive therein a donor transfer absorbent 144 suitably configured/sized for placement within the anode-donor portion 140. Likewise, the cathode-return portion 142 is structured to receive therein a return transfer absorbent 146 suitably configured/sized for placement within the cathode-return portion 142. The transfer absorbents 144, 146 may be attached to their respective portions 140, 142 by a suitable method or apparatus, such as by use of one or more spot welds, for example. In construction of the assembly 100, it can be seen that the release cover 138 is structured for communication with the flexible backing adhesive layer 110 such that the donor transfer absorbent 144 establishes contact with the hydrogel reservoir 134 associated with the anode 104 and the return transfer absorbent 146 establishes contact with the hydrogel reservoir 136 associated with the cathode 106.

In various embodiments, the integrated assembly 100 may include a first reservoir-electrode assembly (including the reservoir 134 and the anode 104) charged with a drug, such as an anaesthetic or a drug combination, such as an anaesthetic and a vasoconstrictor that may function as a donor assembly. The assembly 100 in this embodiment additionally includes a second reservoir-electrode assembly (including the reservoir 136 and the cathode 106) that may function as a return assembly. The assembly 100 includes the reservoir-electrode 104 and the reservoir-electrode 106 mounted on an electrode assembly securement portion 108A of the flexible backing 108. The assembly 100 includes two electrodes, an anode 104 and a cathode 106, each having an electrode surface and an operatively associated electrode trace or lead 112 and 114, respectively. The electrodes 104, 106 and the electrode traces 112, 114 may be formed as a thin coating deposited onto the electrode layer 102 by use of a conductive ink, for example. The conductive ink may include Ag and Ag/AgCl, for example, in a suitable binder material, and the conductive ink may have the same composition for both the electrodes 104, 106 and the electrode traces 112, 114. A substrate thickness for the conductive ink may be in the range of about 0.005 cm to 0.018 cm. In other aspects, the specific capacity of the conductive ink is preferably in the range of about 2 to 120 mA·min/cm², or more preferably in the range of 5 to 20 mA·min/cm². In various aspects, the conductive ink may comprise a printed conductive ink. The electrodes 104, 106 and the electrode traces 112, 114 may be formed in the electrode layer 102 to comprise a stiff portion of the assembly 100.

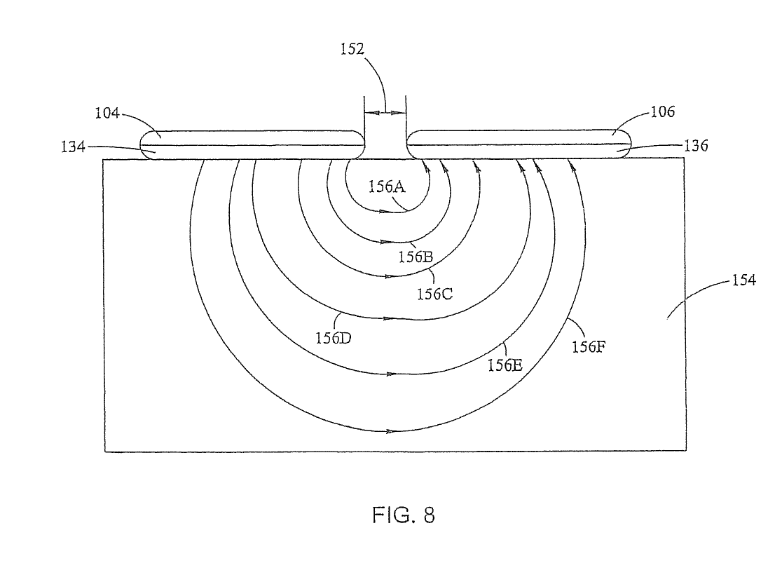

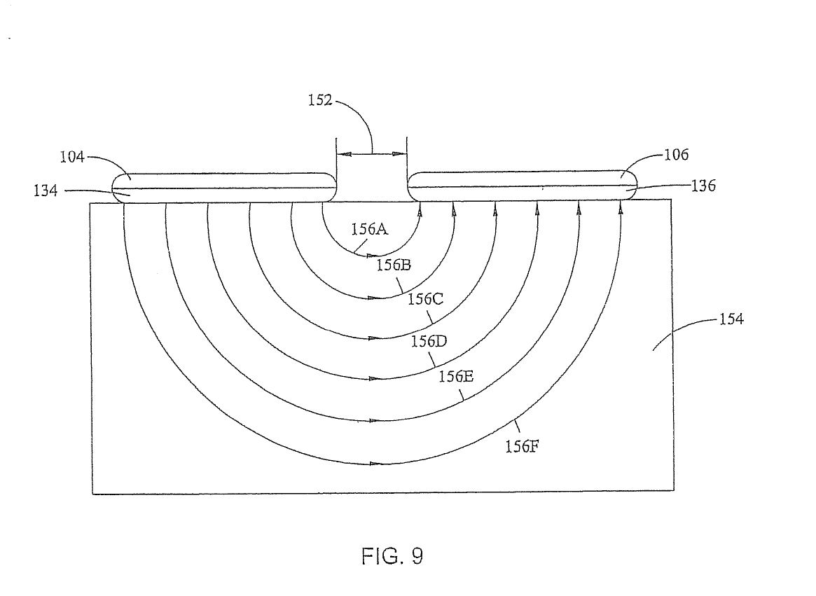

In various embodiments of the integrated electrode assembly, a shortest distance 152 between a surface area of the anode 104/reservoir 134 assembly and a surface area of the cathode 106/reservoir 136 assembly may be in the range of at least about 0.635 cm. Referring now to FIG. 8, for example, it can be seen that inappropriate selection of the distance 152, the geometric configuration of the electrodes 104, 106 (e.g., thickness, width, total surface area, and others), and/or a combination of other factors may result in a substantially non-uniform delivery of a composition between the electrodes through a membrane 154 during operation of the assembly 100. As shown, the delivery of the composition through the membrane is shown schematically by composition delivery paths 156A-156F. In contrast, as shown in FIG. 9, appropriate selection of the distance 152, the geometric configuration of the electrodes 104, 106 (e.g., thickness, width, total surface area, and others), and/or a combination of other factors may result in a substantially uniform delivery of a composition between the electrodes through a membrane 154 as shown by delivery paths 156A-156F. Variations in the conductivity of the membrane and abnormal tissue beneath it may adversely impact the effectiveness and uniformity of delivery of the composition between the electrodes of a device, for example.

In accordance with the discussion above, the electrodes 104, 106 may each be mounted with bibulous reservoirs 134, 136, respectively, formed from a cross-linked polymeric material such as cross-linked poly(vinylpyrrolidone) (“PVP”) hydrogel, for example, including a substantially uniform concentration of a salt, for example. The reservoirs 134, 136 may also include one or more reinforcements, such as a low basis weight non-woven scrim, for example, to provide shape retention to the hydrogels. The reservoirs 134, 136 each may have adhesive and cohesive properties that provide for releasable adherence to an applied area of a membrane (e.g., the skin of a patient). In various embodiments, the strength of an adhesive bond formed between portions of the assembly 100 and the application area or areas of the membrane is less than the strength of an adhesive bond formed between the membrane and the reservoirs 134, 136. These adhesive and cohesive properties of the reservoirs 134, 136 have the effect that when the assembly 100 is removed from an applied area of a membrane, a substantial amount of adhesive residue, for example, does not remain on the membrane. These properties also permit the reservoirs 134, 136 to remain substantially in electrical communication with their respective electrodes 104, 136 and the flexible backing 108 to remain substantially in communication with the printed electrode layer 102.

Portions of the assembly 100, as provided in accordance with certain embodiments of the present invention, may be structured to exhibit flexibility or low flexural rigidity in multiple directions along the structure of the device 100. Working against flexibility of the device 100, however, may be the construction of the comparatively stiffer electrode layer 102, which may include a material such as print-treated polyethylene terephthalate (“PET”), for example, as a substrate. PET is a relatively strong material exhibiting high tensile strength in both the machine and transverse directions and having a flexural rigidity, G=E*δⁿ, which is a function of modulus of elasticity (E) and a power of the thickness (δ) of the material. By way of a hypothetical counter-example, if a substance such as Mylar™, for example, were to be used for both the electrode layer 102 and the flexible backing 108, at least two problems could be presented: (1) the assembly 100 may be too inflexible to fully or effectively adhere to a site of treatment on a membrane, and (2) upon removal from the membrane once treatment is completed, the assembly 100 would require a relatively high level of force, due to the strength of the flexible backing 108, to remove the assembly 100.

Certain embodiments of the present invention provide the flexible backing 108 around the periphery of the stiff electrode layer 102. In certain aspects of particular embodiments, a relatively thin and highly compliant flexible backing composed of about 0.004 inch ethylene vinyl acetate (“EVA”), for example, may be used for the flexible backing 108. This configuration offers a flexible and compliant assembly 100 in multiple planar directions, permitting the assembly 100 to conform to the contour of a variety of membranes and surfaces. In addition, a pressure sensitive adhesive (e.g., polyisobutylene (“PIB”)) may be applied as the transfer adhesive layer 110 to mitigate a potential decrease in flexibility of the flexible backing 108. It can be seen that, in various embodiments, devices constructed in accordance with the present invention permit a degree of motion and flexure during treatment without disrupting the function of the assembly 100. The assembly 100 therefore exhibits low flexural rigidity in multiple directions, permitting conformability of the assembly 100 to a variety of membrane surface area configurations in a manner that is substantially independent of the chosen orientation of the assembly 100 during normal use. In various embodiments, the flexural rigidity of at least a portion of the flexible backing 108 is less than the flexural rigidity of at least a portion of the electrode layer 102.

In general, improvement in certain performance characteristics of certain embodiments of the present invention is realized in minimizing the “footprint” of the assembly 100 when the assembly 100 is applied to a membrane to deliver a composition. As applied herein, the teen “footprint” refers to the portion or portions of the assembly 100 that contact a membrane surface area (e.g., a patient's skin) during operation of the assembly 100. In certain aspects, the surface area of an assembly including the donor electrode 104 and the donor reservoir 134 may be structured to be greater than the surface area of an assembly including the return electrode 106 and the return reservoir 134 to limit the effect of the return assembly on the overall footprint of the assembly 100. In addition, the length of the distance 152 that provides separation between the anode 104 and cathode 106 may also impact the footprint. Furthermore, the size of the electrodes 104, 106 relative to their respective reservoirs 134, 136 may also affect the footprint of the assembly 100. In certain aspects, the reservoirs 134, 136 should be at least substantially the same size as their respective electrodes 104, 106.

It can be appreciated that the inventors have also recognized that once the surface area of the electrode layer 102 is fixed, including configuration of the anode 104 and cathode 106 separation distance 152, the assembly 100 preferably should be sufficiently flexible and adherent for use on a membrane (e.g., a patient's skin). These objectives may depend on the peripheral area of the transfer adhesive layer 110 that surrounds the stiff electrode layer 102. In various embodiments, the width of the peripheral area of the transfer adhesive layer 110 adjacent to one or both of the anode 104 and cathode 106 may be provided as a minimum width 137 (as shown, for example, in FIG. 3). The minimum width 137 may be structured, in certain aspects, in the range of at least about 0.953 cm. In turn, these objectives depend on the aggressiveness of the transfer adhesive layer 110 and the flexible backing 108, which is preferably flexible and compliant as a function of the strength (e.g., modulus of elasticity) and thickness of the flexible backing 108. Any sufficiently thin material may be flexible (such as ultra-thin PET, for example), but another problem arises in that the transfer adhesive layer 110 and the flexible backing 108 preferably are capable of removal from a membrane with minimum discomfort to a patient, for example. Consequently, a compliant (i.e., low strength) flexible backing 108 may be employed while maintaining adequate strength for treatments using the assembly 100.

The footprint area of the assembly 100 may be preferably in the range of about 3 cm² to 100 cm², more preferably in the range of about 5 cm² to 60 cm², and most preferably in the range of about 20 cm² to 30 cm². In addition, the total electrode 104, 106 area may be in the preferred range of about 2 cm² to 50 cm², or more preferably in the range of about 3 cm² to 30 cm², and most preferably in the range of about 4 cm² to 40 cm², respectively. In other aspects, the ratio of the area of each reservoir 134, 136 to its corresponding electrode 104, 106 may be, for example, in the range of about 1.0 to 1.5. In one operational example, the total contact area for the electrodes 104, 106 is about 6.3 cm² and the total reservoir 134, 136 contact area is about 7.5 cm². In other aspects, the flexible backing adhesive layer 110 for the printed electrode layer 102 may have a thickness in the range of, for example, about 0.004 cm to about 0.013 cm. The flexible backing 108 may be comprised of a suitable material such as, for example, EVA, polyolefins, polyethylene (“PE”) (such as, for example, low-density polyethylene (“LDPE”), polyurethane (“PU”), and/or other similarly suitable materials.

According to other aspects of certain non-limiting embodiments according to the present invention, the ratio of total electrode surface area to total footprint area may be in the range about 0.1 to 0.7, or preferably about 0.24. In certain aspects, the ratio of donor electrode 104 surface area to return electrode 106 surface area may be in the range of about 0.1 to 5.0, or preferably about 1.7. In still other aspects, the ratio of donor reservoir 134 thickness to return reservoir 136 thickness may be in the range of about 0.1 to 2.0, or more preferably about 1.0.

FIGS. 5B and 5C each show the layering of elements of the electrode assembly 100 as shown in FIG. 5A. In FIGS. 5B and 5C, it can be seen that the thickness of layers is not to scale and adhesive layers are omitted for purposes of illustration. FIG. 5B shows a cross section of the anode electrode 104/reservoir 134 assembly and the cathode electrode 106/reservoir 136 assembly. The anode 104 and the cathode 106 are shown layered on the printed electrode layer 102. The anode reservoir 134 and the cathode reservoir 136 are shown layered on the anode 104 and the cathode 106, respectively. FIG. 5C is a cross-sectional view through the anode 104, the anode trace 112, and the anode reservoir 134. The anode 104, the anode trace 112 and a sensor trace 130 are layered upon the electrode layer 102. The anode reservoir 134 is shown in communication with the anode 104. The tab stiffener 124, which may be composed of an acrylic material, for example, is shown attached to the tab end 116 of the assembly 100. In addition, the sensor trace 130 may be located at the tab end 116 of the electrode assembly 100.

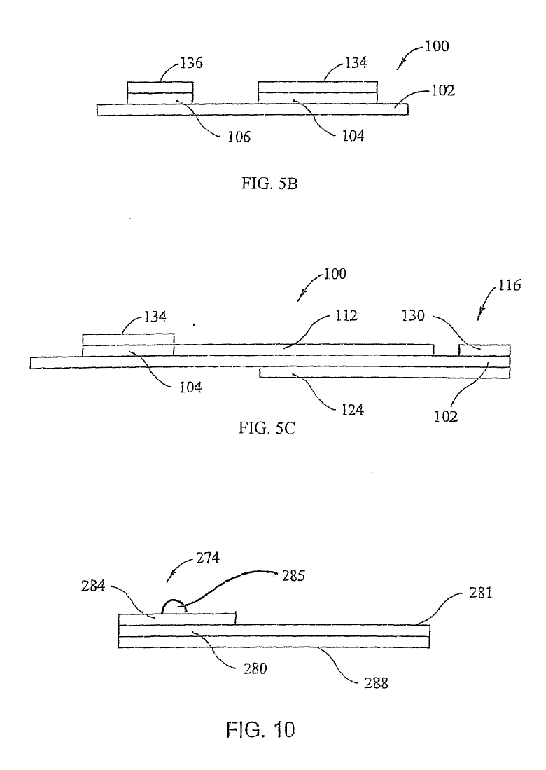

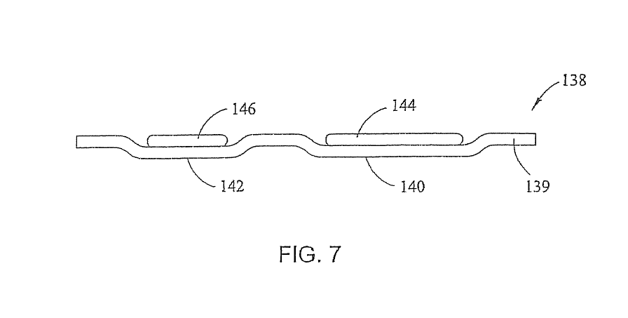

In other embodiments of the integrated electrode assembly, FIGS. 6 and 7 show schematically the release cover 138 structured for use with various devices, electrode assemblies and/or systems of the present invention. The release cover 138 includes a release cover backing 139, which includes an anode absorbent well 140 and a cathode absorbent well 142. In Platform I, a nonwoven anode absorbent pad is contained within the anode well 140 as the transfer absorbent 144, and a nonwoven cathode absorbent pad is contained within the cathode well 142 as the transfer absorbent 146. In use, the release cover 138 is attached to the electrode assembly 100 so that the anode absorbent pad 144 and the cathode absorbent pad 146 substantially cover the anode reservoir 134 and the cathode reservoir 136, respectively. The anode absorbent pad 144 and the cathode absorbent pad 146 may each be slightly larger than their corresponding anode reservoir 134 or cathode reservoir 136 to cover and protect the reservoirs 134, 136. The anode absorbent pad 144 and the cathode absorbent pad 146 may also be slightly smaller than the anode absorbent well 140 and the cathode absorbent well 142, respectively. In various embodiments, one or more indicia 220 (e.g., a “+” symbol as shown) may be formed on at least a portion of the flexible backing 108 of the assembly 100 adjacent to the anode well 140 and/or the donor well 142. It can be appreciated that the indicia 220 may promote correct orientation and use of the assembly 100 during performance of an iontophoretic procedure, for example.

The anode absorbent pad 144 and the cathode absorbent pad 146 may be attached to the backing 139 of the release cover 138 by one or more ultrasonic spot welds such as welds 222, 224, 226, for example, as shown in FIG. 7. The welds 222, 224, 226 may be substantially uniformly distributed in areas of connection between the non-woven fabric pads 144, 146 and the wells 140, 142, respectively.

In various embodiments, the donor electrode reservoir 134, for example, may be loaded with an active ingredient from an electrode reservoir loading solution by placing an aliquot of the loading solution directly onto the hydrogel reservoir and permitting the loading solution to absorb and diffuse into the hydrogel over a period of time. FIG. 10 illustrates this method for loading of electrode reservoirs in which an aliquot of loading solution is placed on the hydrogel reservoir for absorption and diffusion into the reservoir. FIG. 10 is a schematic cross-sectional drawing of an anode electrode assembly 274 including an anode 280 and an anode trace 281 on a backing 288 and an anode reservoir 284 in contact with the anode 280. An aliquot of a loading solution 285, containing a composition to be loaded into the reservoir 284 is placed in contact with reservoir 284. Loading solution 285 is contacted with the reservoir 284 for a time period sufficient to permit a desired amount of the ingredients in loading solution 285 to absorb and diffuse into the gel reservoir 284. It can be appreciated that any suitable method or apparatus known to those in the art may be employed for loading the reservoir 284 with a composition.

In use, electrode reservoirs described herein can be loaded with an active ingredient from an electrode reservoir loading solution according to any method suitable for absorbing and diffusing ingredients into a hydrogel. Two possible methods for loading a hydrogel include, without limitation, placing the hydrogel in contact with an absorbent pad material, such as a nonwoven material, into which a loading solution containing the ingredients is absorbed. A second loading method includes the steps of placing an aliquot of the loading solution directly onto the hydrogel and permitting the loading solution to absorb and diffuse into the hydrogel over a period of time.

In applying the first method just mentioned to the electrode assembly 100, for example, the loading solution containing ingredients to be absorbed and diffused into the respective anode reservoir 134 and cathode reservoir 136 are first absorbed into the nonwoven anode absorbent pad 144 and nonwoven cathode absorbent pad 146, respectively. When a release cover thus loaded is connected to electrode assembly 100, the ingredients therein desorb and diffuse from the absorbent pads 144 and 146 and into the respective reservoirs. In this case, absorption and diffusion from the reservoir cover into the reservoirs has a transfer efficiency of about 95%, requiring that about a 5% excess of loading solution be absorbed into the absorbent pads. Despite this incomplete transfer, the benefits of this loading process, as compared to placing a droplet of loading solution onto the reservoirs and waiting between about 16 and 24 hours or so for the droplet to immobilize and absorb, can be significant because once the release cover is laminated onto the electrode assembly, the assembly can be moved immediately for further processing and placed in inventory. There is no requirement that the assembly is kept flat and immobile while awaiting completion of absorption and/or diffusion.

The transfer absorbents 144 and 146 are typically a nonwoven material. However, other absorbents may be used, including woven fabrics, such as gauze pads, and absorbent polymeric compositions such as rigid or semi-rigid open cell foams. In the particular embodiments described herein, as noted above, the efficiency of transfer of loading solution from the absorbent pads of the release cover to the reservoirs is about 95%. It will be appreciated by those skilled in the art that transfer efficiency will vary depending on the composition of the absorbent pads and the reservoirs as well as additional physical factors including, without limitation, the size, shape, and thickness of the reservoirs and absorbent pads and the degree of compression of the absorbent pads and reservoirs when the release cover is affixed to the electrode assembly. The transfer efficiency for any given release cover-electrode assembly combination can be readily determined empirically and, therefore, the amount of loading solution needed to fully load the reservoirs to their desired drug content can be readily determined to target specifications.

As discussed above, FIG. 10 illustrates the second method described above for loading of electrode reservoirs, wherein an aliquot of loading solution is placed on the hydrogel reservoir for absorption and diffusion into the reservoir. The transfer absorbents 144, 146 typically need not be included in the release cover for electrode assemblies having reservoirs loaded by this method.

The Platforms II and III embodiments differ from Platform I in that the drug or drug combination is drop loaded into the anode reservoir.

To facilitate removal of the release cover 138 from the electrode assembly 100, portions of the backing 139 in communication with the transfer adhesive 110 when the release cover 138 is attached to the electrode assembly 100 may be treated with a release coating, such as a silicone coating, for example.

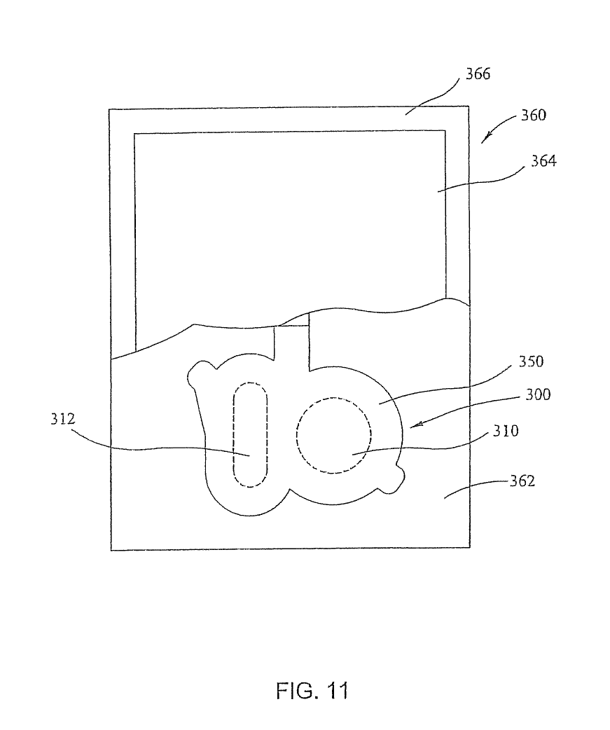

FIG. 11 is a breakaway schematic representation of the electrode assembly 300 within a hermetically sealed packaging 360. Packaged electrode assembly 300 is shown with release liner 350 in place and anode 310 and cathode 312 are shown in phantom for reference. Hermetically sealed packaging 360 is a container that is formed from a first sheet 362 and a second sheet 364, which are sealed along seam 366. In use, sheets 362 and 364 are sealed together to form a pouch after electrode assembly 300 is placed on one of sheets 362 and 364.

Other techniques well-known to those skilled in the art of packaging may be used to form a hermetically sealed package with an inert atmosphere. In one embodiment, the moles of oxygen in the inert gas in the sealed pouch is limited, by controlling the oxygen concentration in the inert gas and by minimizing the internal volume, or headspace, of the package, to be slightly less than the amount of sodium metabisulfite in the epinephrine-containing reservoir needed to react with all oxygen in the package. Electrode assembly 300 is then inserted between sheets 362 and 364, an inert gas, such as nitrogen is introduced into the pouch to substantially purge air from the pouch, and the hermetically sealed packaging 360 is then sealed. The hermetically sealed packaging 360 may be sealed by adhesive, by heat lamination or by any method know to those skilled in the art of packaging devices such as electrode-assembly 300.

### Active and Passive Ingredients

For the indications of use described herein, the active ingredients are an anaesthetic and optionally a vasoconstrictor. The precise amounts of each active ingredient will vary according to recognized pharmacological doses for the type of procedure, the depth of the dermal layers affected and the duration of analgesia required. As in any medical procedure involving anaesthesia, the medical professionals performing and assisting in the procedure would closely monitor the patient and provide additional levels as needed. Adjustments in the amount of active ingredient delivered prior to a procedure which may be required due to differences in the age, size and sensitivity of the individual patient are within the skill of the medical professionals performing the procedures.

For those indications where systemic delivery of the anaesthetic is to be avoided or minimized, for example, where the goal is analgesia of the dermal layers of the skin, a vasoconstrictor is combined with the anaesthetic as the active ingredient, with major amounts of anaesthetic relative to minor amounts of the vasoconstrictor. In those indications where systemic delivery of anaesthesia is desired, the vasoconstrictor is preferably eliminated or the relative amount of vasoconstrictor is significantly reduced.

Studies 1 and 2

Two studies (Studies 1 and 2) were done to determine the efficacy of including a vasoconstrictor, such as epinephrine, together with lidocaine as the anaesthetic in the Electrotransport device. The results from Study 1 showed that iontophoretic treatments using patches containing 10% lidocaine and 0.1% epinephrine provided significantly greater anaesthesia than equivalent iontophoretic treatments using patches containing 10% lidocaine alone. In addition, vein diameters and ease of cannulation scores were not significantly different between treatment using patches with and without epinephrine.

In Study 2, the degree of anaesthesia was also higher in patches containing 0.1% epinephrine in addition to 10% lidocaine, and pain upon patch removal and sensation from iontophoresis were not different in 10% lidocaine patches with or without 0.1% epinephrine. Therefore, the inclusion of 0.1% epinephrine to the 10% lidocaine patches contributes to the effectiveness of anaesthesia at optimized operating parameters without affecting vein size, pain upon patch removal, or sensation from iontophoresis.

The anaesthetic in combination with a vasoconstrictor, for example, Lidocaine HCl and epinephrine bitartrate, are used in several of the examples herein to elicit a desired pharmacological response. Chloride ions are useful in preventing electrode corrosion. If the counterion of lidocaine, for example, is not chloride, a corrosion-inhibiting amount of another counterion may be present in lieu of, or in addition to, the unloaded reservoir or in the chloride ions to prevent corrosion of the electrode. If more than one counterion is present, such as in the case where more than one drug is loaded and each drug has a different counterion, it may be preferable to include sufficient amounts of both counterions in the reservoir to prevent electrode corrosion. It should be noted that in the examples provided below, the amount of epinephrine bitartrate loaded into the gel is not sufficient to cause corrosion.

Lidocaine and epinephrine are both positively charged and delivered simultaneously from the circular drug reservoir. Lidocaine stabilizes the neuronal membrane by inhibiting the ionic fluxes required for the initiation and conduction of nerve impulses, thereby effecting local anaesthetic action. Because of its vasoconstrictor activity, which decreases the rate of removal of Lidocaine from the site of administration, epinephrine increases the depth and duration of the anaesthesia. In the absence of the vasoconstrictor, the rate of removal of the anaesthetic from the site of administration is more rapid, thereby increasing its systemic penetration.

Calculations have also been done to determine the theoretical amount of drug (both lidocaine and epinephrine) that is transported into the skin during the iontophoresis process. The amount of drug delivered during the iontophoresis process is dependent primarily on (1) the concentration of drug in the formulation relative to the concentration of other ionic competing species, and (2) the total current delivered during the iontophoresis process. Using a 10% lidocaine solution delivered with a maximum total charge of 17 mA·min, the total amount of lidocaine delivered is 547 μg. A similar calculation for epinephrine shows that, using a 0.1% epinephrine solution, the estimated total delivery of epinephrine is 6.2 μg. In vivo experiments examining delivery of radiolabeled drug into anesthetized guinea pigs shows that these theoretical estimates are consistent with actual drug delivery (467 μg lidocaine and 2.4 μg epinephrine were delivered per patch in guinea pigs). These theoretical values are orders of magnitude less than the maximum recommended dose of lidocaine (300 mg) and the maximum recommended dose of epinephrine codelivered with 300 mg of lidocaine (150 μg).

Taking together the data obtained from these clinical studies and that from theoretical calculations of drug delivery, it was determined that 10% lidocaine and 0.1% epinephrine for 10 minutes at 17 mA·min was optimally effective at providing anaesthesia with minimal systemic side effects.

Although Lidocaine is a common topical anaesthetic, other useful topical (surface and/or infiltration) anaesthetics may be used in the described system. These anaesthetics include, without limitation, salts of: amide type anaesthetics, such as bupivacaine, butanilicaine, carticaine, cinchocaine/dibucaine, clibucaine, ethyl parapiperidino acetylaminobenzoate, etidocaine, lidocaine, mepivicaine, oxethazaine, prilocaine, ropivicaine, tolycaine, trimecaine and vadocaine; ester type anaesthetics, including esters of benzoic acid such as amylocaine, cocaine and propanocaine, esters of metaminobenzoic acid such as clormecaine and proxymetacaine, esters of paraminobenzoic acid (PABA) such as, amethocaine (tetracaine), benzocaine, butacaine, butoxycaine, butyl aminobenzoate, chloroprocaine, oxybuprocaine, parethoxycaine, procaine, propoxycaine and tricaine; and miscellaneous anaesthetics, such as, bucricaine, dimethisoquin, diperodon, dyclocaine, ethyl chloride, ketocaine, myrtecaine, octacaine, pramoxine and propipocaine.

Of the topical anaesthetics, salts of bupivacaine, butacaine, chloroprocaine, cinchocaine, etidocaine, mepivacaine, prilocaine, procaine, ropivacaine and tetracaine (amethocaine) might be considered by some to be more clinically relevant than other anaesthetics listed above, though not necessarily more effective. Bupivacaine is the most frequently used agent. Certain other features of each of the compounds listed above may make any particular compound more or less suited to iontophoretic delivery as described herein. For example, use of cocaine may be contra-indicated because of its cardiovascular side effects. Bupivacaine, butacaine, chloroprocaine, cinchocaine, etidocaine, mepivacaine, prilocaine, procaine, ropivacaine and tetracaine (amethocaine) may be preferred as substitute for lidocaine because the all have similar pKs of about 8 or >8, meaning they will ionize under the same conditions as lidocaine. Iontophoresis in vitro across human skin has shown that bupivacaine and mepivacaine show a similar cumulative delivery as lidocaine, while etidocaine, prilocaine and procaine have shown slightly greater delivery. Chloroprocaine, procaine and prilocaine have similar relatively short duration effects (<2 hr) whereas bupivicaine, etidocaine, and mepivacaine have effects lasting 3-4 hr. These times are approximately doubled when a vasoconstrictor, such as epinephrine is used in conjunction with these anaesthetics. The duration of the action of the local anaesthetic is dependent upon the time for which it is in contact with the nerve. This duration of effect will depend on the physiochemical and pharmacokinetic properties of the drug. Hence, any procedure that can prolong contact between the therapeutic agent and the nerve, such as co-delivery of a vasoconstrictor with the anaesthetic, will extend the duration of action.

A factor in the choice of the anaesthetic is that ester-based anaesthetics based on PABA are associated with a greater risk of provoking an allergic reaction because these esters are metabolized by plasma cholinesterase to yield PABA, a known allergen. For this reason, amide anaesthetics might be preferred and molecules such as chloroprocaine, and procaine would not be viewed as first-line replacements for lidocaine. Because bupivacaine, etidocaine, mepivacaine, ropivicaine and prilocaine are amide anaesthetics with similar physiochemical properties and clinical effects as lidocaine, they may be preferred by some as substitutes for lidocaine. A secondary issue with prilocaine is that although it is generally considered to be the safest of the amide anaesthetics, one of its metabolites (o-toluidine) has been associated with increased risk of methemoglobinemia and cyanosis as compared to the other amide anaesthetics.

These drugs can be delivered as racemates or enantiomers. The enantiomers have different pharmacokinetic profiles and appear to exert slightly different pharmacological effects in particular, lower risk profiles. Hence iontophoretic delivery of specific enantiomers appears to be advantageous in those situations requiring prolonged, continuous application, such as in the treatment of chronic refractory pain resulting from any cause, including neuropathic pain, cancer, and diabetic neuropathy, neuropathy of shingles, post herpetic neuralgia and trigeminal neuralgia.

Each of the anaesthetics listed above have varying degrees of vasoconstrictor activity. Therefore, optimal concentrations of the anaesthetic and the vasoconstrictor will vary depending on the selected local analgesic. However, for each local anaesthetic, optimal effective concentration ranges can be readily determined empirically by functional testing.

In all Platforms described herein, the donor (anode) reservoir also includes a salt, preferably a fully ionized salt, for instance a halide salt such as sodium chloride in a concentration of from about 0.001 wt. % to about 1.0 wt. %, preferably from about 0.06 wt. % to about 0.09 wt. %. The salt content is sufficient to prevent electrode corrosion during manufacture and shelf-storage of the electrode assembly. These amounts may vary for other salts in a substantially proportional manner depending on a number of factors, including the molecular weight and valence of the ionic constituents of each given salt in relation to the molecular weight and valence of sodium chloride. Other salts, such as organic salts, are useful in ameliorating the corrosive effects of certain drug salts. Typically the best salt for any ionic drug will contain an ion that is the same as the counter ion of the drug. For instance, acetates would be preferred when the drug is an acetate form. However, the aim is to prevent corrosion of the electrodes.

Sodium metabisulfite may be added to the donor reservoir to scavenge oxygen. The amount of sodium metabisulfite added is not substantially in excess of the amount needed to scavenge all oxygen from the packaged reservoir for a given time period to minimize the formation of the adduct epinephrine sulfonic acid, and other decomposition products. For example, the donor hydrogel may contain less than about 110%, for example about 101%, of the amount of sodium metabisulfite equal to a minimal amount of sodium metabisulfite needed to scavenge substantially all oxygen in the packaged donor hydrogel. The amount of sodium metabisulfite needed to scavenge oxygen in the packaged donor hydrogel for any given amount of time can be calculated from the amount of oxygen present within the package in which the donor hydrogel is hermetically sealed. Alternately, the optimal amount of sodium metabisulfite can be titrated by determining the amount of sodium metabisulfite at which production of the oxidation products of epinephrine, due to its reaction with oxygen, such as adrenolone or adrenochrome, and epinephrine sulfonic acid essentially stops.

The return (cathode) reservoir may be a hydrogel with the same or different polymeric structure as the donor (anode) hydrogel and typically contains a salt such as sodium chloride, a preservative and, optionally, a humectant. Depending upon the ultimate manufacturing process, certain ingredients may be added during cross-linking of the hydrogel reservoir, while others may be loaded with the active ingredients. Nevertheless, it should be recognized that irrespective of the sequence of addition of ingredients, the salt must be present in the reservoir adhering to the electrode and substantially evenly distributed therethrough prior to the loading of the active ingredient(s) or other ingredient that causes formation of concentration cells.

An exemplary anode reservoir composition may be prepared for Platform I using the PVP, phenonip, NaCl, and purified water. The anode gel reservoirs were loaded with a drug loading solution which was accomplished by placing 0.32 ml aliquots of drug loading solution on the reservoirs and the solution was then permitted to absorb and diffuse into the reservoir.

An exemplary cathode reservoir composition may be prepared for Platform I using the PVP, phenonip, NaCl, and purified water. The cathode gel reservoirs were loaded with an electrolyte solution which was accomplished by placing 227 mg electrolyte solution on the reservoirs and the solution was then permitted to absorb and diffuse into the reservoir.

Within-lot variation in solution doses and composition typically is +5%, but has not been analyzed statistically.

In another embodiment, unloaded gel reservoirs within an integrated patch assembly for any of Platforms IIA, IIB or III were prepared using the PVP, phenonip, NaCl, and purified water. The unloaded anode gel reservoirs were placed on Ag/AgCl anodes and 0.32 ml aliquots of drug loading solution were placed on the reservoirs and were permitted to absorb and diffuse into the reservoir.

### Measurements of Dermal Analgesia

Prior to evaluating the performance of the Electrotransport System in puncture-type procedures or more involved dermal procedures, such as incisional or excisional procedures or laser removal of superficial skin lesions, an understanding of the quantitative performance limitations of the system was desired. A study was therefore designed to evaluate the depth of anaesthesia penetration into the skin in normal human volunteers, and to assess the characteristics of the effect over an extended duration.

Aesthesiometers are used to test the threshold for the tactile receptors in the skin. They are widely used in hand surgery and rehabilitation to detect and monitor peripheral nerve function or results of nerve repair. They are also used to objectively determine touch thresholds, screening for peripheral nerve impairment, determining spatial extent and degree of nerve impairment, and detecting changes in neurological status. For example, aesthesiometers can be used to determine the location and delineation of areas of analgesia, or absence of pain and touch sensitivity, as well as areas of hyposthenia, that is reduced pain or touch sensitivity, of the skin of a person, for example, as is shown below. A very common type of aesthesiometer is a filament aesthesiometer in which a filament is pressed perpendicularly to the skin and the applied pressure is measured to determine tactile thresholds. Other aesthesiometers apply pressure in different ways, such as air pressure to measure tactile thresholds.

Around 1900, Max von Frey discovered that horse hairs tended to apply a single downward force that was not proportional to bending in that horse hairs could be used to measure anaesthesia. In contrast, for the common spring, the downward force is directly proportional to the bending. Modern filament aesthesiometers use monofilaments, such as nylon monofilaments rather than horse hair. Nylon monofilament was not invented until WWII. Sidney Weinstein immediately thereafter employed the nylon monofilament to produce a set of 20 diameter-varying and length-constant monofilaments. These monofilaments produce a characteristic force perpendicular to the contacting surface. The characteristic forces for his set of monofilaments were published, and that set of nylon monofilaments on plexiglass handles is known today as the Semmes-Weinstein Aesthesiometer (SWA). “Aesthesiometer filaments,” collectively refer to any filament, such as, without limitation, horse hair or nylon monofilament, used in an aesthesiometer.

Aesthesiometer filaments will produce varying sensations of touch when applied to the skin. By applying an increasing axial force along the filament, with one end of the filament engaged with and perpendicular to the patient's skin, the filament will apply an increasing force on the patient's skin. As the monofilaments are placed on the skin, they begin to bend. This force can be so small that tactile receptors cannot sense it. When the column buckling stress of the filament is reached, the filament will bend sideways in an arch as the force and pressure applied to the patient by the filament decreases from a predetermined maximum value.

By standardizing the length, diameter and modulus of the filament, a standardized present maximum force can be repeatedly applied to a patient at the point where the filament initiates buckling. Each monofilament number corresponds to level of force provided by that monofilament. The common monofilament is a single strand of nylon, which has the property of producing a characteristic downward force when buckled on a surface. The downward force does not depend on the degree of bend of the monofilament. Once in contact with the skin, the monofilament starts to bend and reaches a force maximum that is not exceeded with further bending. The actual force varies around the characteristic force for that monofilament. Equations predict the characteristic force from the diameter and the length of the monofilament.

In embodiments of the present invention, aesthesiometer measurements were used to determine the level of analgesia achieved by the described iontophoretic devices. In particular embodiments of the invention, an amount of a vasoconstrictor and an anaesthetic are pre-loaded in an iontophoretic device. Drug delivery is then electrically assisted for a period of time, producing at least a 50% reduction of dermal sensitivity to an applied force as measured by a filament aesthesiometer and producing a hedonic score (described below) of greater than about −1.5 on a visual analogue scale (described below) ranging from −10 to 10. In these embodiments, the vasoconstrictor is delivered in an amount that will not result in skin necrosis, i.e., will not necrotize the skin.

In particular embodiments, the period of time of electrically driven delivery ranges from 1 to 30 minutes, and more preferably from 5 to 20 minutes. In one embodiment, the period of time of electrically driven delivery is about 20 minutes. In yet other embodiments, the electrical assistance is provided by using current densities ranging from 0.1 to 4.2 mA·min/cm², preferably between and including 2.4 to 3.4 mA·min/cm².

### Depth and Duration Study

A study was conducted to assess the depth and duration of dermal anaesthesia produced by an iontophoresis drug delivery system delivering a drug formulation including 10% lidocaine anaesthetic and 0.1% epinephrine vasoconstrictor and producing an approximately 5 cm² region of local anaesthesia on treated skin. The iontophoresis drug delivery system was constructed generally as shown in the attached FIGS. 2, 3, 4, 5, 5A-C, 6A-C, 7, and 7A, and as described in the foregoing text describing the device illustrated in those figures.

A primary objective of the study was to quantify the depth to which clinically meaningful anaesthesia penetrates the skin immediately after treatment with the drug delivery system compared with a suitably designed placebo. A secondary objective of the study was to quantify the depth to which the sensation (such as pressure) is eliminated after treatment with the drug delivery system compared to placebo (which was an identical drug delivery device loaded with 0% lidocaine and 0.1% epinephrine), and to measure the depth of anaesthetic effect over time from both pain and sensory perspectives.

Pain threshold depth (“PD”) is depth at which a patient senses pain upon the insertion of an 18 gauge needle that a rate of 0.2 mm per second. The patient pushes a button, which automatically records the depth. The needle stops movement. The maximum value was preset to 25 iron. The Sensory Penetration Depth is the sensory threshold depth (“SD”) at which a patient senses a feeling of pressure upon the insertion of an 18 gauge needle that a rate of 0.2 mm per second. The patient pushes a button, which automatically records the depth and continues to the point of PD.

Pain threshold depth measurements indicated that clinically meaningful anaesthesia penetrated significantly farther into the skin after treatment with the Electrotransport System compared with placebo. The study confirmed that the iontophoresis electrode assembly produced clinically acceptable depth and duration of anaesthesia on the treated skin site. A purpose of the study was to develop quantitative insight on the performance of the tested iontophoresis system and drug formulation. The depth to which all sensation was eliminated was also significantly higher with the Platform I Electrotransport System compared with placebo. The difference between mean anaesthesia penetration depths for active and placebo iontophoresis treatments (6.37 mm vs. 3.09 mm) at T=0 is considered clinically meaningful. Since the duration of the anaesthesia effect exceeded the 60-minute post-treatment measurement interval with the active treatment, the durability of the anaesthesia effect with the active treatment also was significant. Pain threshold depth and sensory threshold depth were maintained throughout the measurement period, demonstrating the durability of the effect. No safety issues were identified during the study.

### Indications for Use of the Electrotransport System for Electrically Assisted Delivery

The integrated electrode assembly described herein can be used to deliver local anaesthesia for a wide variety of indications. Examples include puncture-type procedures involving puncturing a patient's skin with a needle or a cannula, procedures involving excisions or incisions with a blade, scalpel, razor, or looped Curette, procedures involving the laser removal of skin lesions, and procedures involving skin scraping, or abrasion by hard particle ablation, “sanding” or laser ablation, or with a blade, scalpel, razor or a looped Curette.

The Electrotransport device may also be used to deliver anaesthetic for treatment of chronic refractory pain resulting from any cause, including, for example, neuropathic pain, cancer, and diabetic neuropathy, neuropathy of shingles, post herpetic neuralgia and trigeminal neuralgia. As described above, for this indication, the combination of a vasoconstrictor with the anaesthesia may not be indicated because the management of pain caused by some lesions would be enhanced by some systemic lidocaine delivery. Subcutaneous infusions of anaesthesia are given for many causes of neuropathic pain including Shingles or diabetic painful neuropathy, plexopathy (shoulder pain from brachial plexus pathology) and neuropathic pain from spinal cord injury, temporomandibular joint dysfunction (TMJ) or trigeminal neuralgia. Patients with refractory pain from a malignancy have lesions that may be managed with the treatment described herein.

Puncture-type procedures include, for example, venipuncture for taking small samples of blood for testing or larger amounts for blood donation, intravenous cannulation (IV cannulation), injections, epidural and lumbar punctures and the administration of regional nerve blocks, needle aspirations, body piercings and tattoo applications. While many individuals undergoing a routine venipuncture procedure wherein blood is drawn with a small gage needle from the dorsum or antecubital fossae of the arm would not need prior application of a local anaesthetic, other individuals are particularly sensitive to the pain caused by even a simple puncture. The injection of certain substances, for example botox and cortisone injections and immunizations containing albumin, can be very painful and/or irritating due to the nature of the substance, and differences in concentration and pH of the normal chemical environment in contact with the tissue, and the size of the needle used for the injection. The electrically assisted delivery of a local anaesthetic as taught herein, can precede injection of local anaesthetic as for example a few cubic centimeters of 2% lidocaine, which would profuse into the blood stream and tissues.

Further, some areas of the body are more sensitive than others. Puncture-type procedures, for example, in the hands or feet, for example, are painful.

In punctures where it is important for the patient to be still to avoid injury (e.g., lumbar and epidural punctures), a local anaesthetic may be required prior to the puncture to ensure that the patient does not move in reaction to the pain from the puncture. The anaesthetics most commonly used for lumbar and epidural punctures and regional nerve blocks include lidocaine, bupivacaine, prilocaine and ropivacaine.

In venipuncture, the choice of vein for needle insertion is generally left to the phlebotomist. The procedure involves palpitating the vein to assess its suitability and depth, cleaning and drying the area, then, applying the patch, with the anode circle centered around the point of planned needle insertion. The current is applied as described above. For IV cannulation, the choice vein or artery may be dictated by other concerns, but the procedure for locating the vein or artery, cleaning the site and applying the appropriate Platform is the same as the procedure for venipuncture.

The examples above demonstrated that the analgesic effect obtained by use of the Electrotransport System obtain to depths of approximately 10-11 mm and for periods of time as long as about 10 to 60 minutes. By adjusting the current density, the length of time the anaesthesia is applied and the strength of the anaesthesia according to known pharmacologic activity associated with different anaesthetics, the analgesic effect can be controlled so that the full depth of the individual's dermal layers in the target location is anaestitized for the duration of the procedure.

### Pharmacokinetic Study

A series of studies was done to test the absorption of the anaesthetic (lidocaine) and vasoconstrictor (epinephrine) using Platform III. The studies, which were done using adult and pediatric volunteers, demonstrated that plasma levels of lidocaine after treatment were below the concentrations required to achieve systemic therapeutic or adverse side effects. The Electrotransport System used a small electric current to deliver lidocaine and epinephrine into the skin in the vicinity of pain receptors and nerve endings.

Information derived from diverse formulations, concentrations and usage revealed that lidocaine was completely absorbed following parenteral administration. Its rate of absorption is dependent upon various factors such as the site and route of administration, and the presence or absence of a vasoconstrictor agent.

The highest lidocaine blood levels are obtained following intercostal nerve block (aside from intravascular administration) and the lowest blood levels are obtained after iontophoretic administration as described herein. Thus, an advantage of the Electrotransport System is the significant reduction of the anaesthetic in the blood stream, thereby avoiding unwanted systemic consequences in those indications where only local anaesthesia is needed. Examples include indications involving puncture-type procedures, incisions and excision procedures, laser treatments and skin surface removal procedures affecting only the dermal layers. Other procedures, such as pain management, particularly the management of neuropathic pain, would benefit from some systemic penetration. The devices described herein would control the amount of active ingredients delivered in this application to limit the system concentration to be within the therapeutic window and be able to minimize toxic effects.

### Effectiveness of the Electrotransport System in Various Indications

Studies were done to evaluate the effectiveness of the Electrotransport System in various indications.

Study 3

In one randomized, double-blind, placebo-controlled, parallel-group study, 48 adult subjects were evaluated for patch removal pain (PRP) and the degree of dermal anaesthesia prior to venipuncture or IV cannulation compared to placebo (no current) treatment. Each subject received treatment with Platform I. The results demonstrated that treatment with Platform I of the Electrotransport System provided better anaesthesia at the antecubital site and on the hand dorsum than the placebo.

Study 4

In a second prospective, placebo-controlled, double-blind study, 20 adult subjects were evaluated to quantify the depth to which clinically meaningful anaesthesia penetrated the skin and the depth to which sensation was eliminated after treatment with the Platform III Electrotransport System and a placebo. In addition, the depth of the anaesthetic effect was measured over time from both pain and sensory perspectives. The average pain threshold depth (PD) and the average sensory penetration depth (SD), described above, immediately after patch removal were statistically significantly greater for the Electrotransport System treatment than for the placebo treatment. For the Electrotransport System treatment, PD increased 60 minutes later, demonstrating the durability of the treatment effect. Average pain threshold depth (PD) at T=0 was 6.37 mm, an increase of 3.28 mm from placebo treatment at the same time point. This difference was statistically significant (p<0.0001).

Average sensory penetration depth (SD) at T=0 was 3.90 mm, an increase of 2.55 mm from placebo, also a statistically significant difference (p<0.0001. Neither cutaneous perception of pain (CP), vascularity in the region (EI), side of treatment, nor skin thickness had a significant effect on the measurements at T=0. Pain threshold depth increased to an average depth of 10.68 mm at T=60 (a 7.33 mm increase from placebo), demonstrating durability of the effect.

Study 5

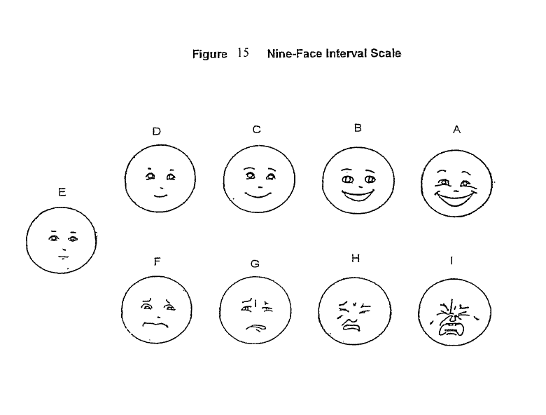

In another randomized, double-blind, placebo-controlled, study, 48 children were stratified by age group (5 to 7 years, 8 to 11 years, and 12 to 18 years) to compare the efficacy of the Electrotransport System with placebo (no current) in providing local dermal anaesthesia prior to venipuncture. Based on scaled scoring using one or both of a Nine Face Integrated Scale and a Visual Analog Scale, children treated with the Platform III Electrotransport System experienced significantly less pain during the venipuncture procedure than did subjects treated with the placebo patch for all age groups in these studies. The length of this scale is 10 cm. The Nine-Face Interval Scale, illustrated in FIG. 15, scores the occurrence and extent of pain experienced by children as assessed by their parent(s)/guardian(s). The Visual Analogue Scale (VAS) is a horizontal liner scale where the lowest value represents the least pain or no pain and the highest value represents the most pain. Units are from 0 to 10.

Multiple clinical studies were conducted to demonstrate that the Electrotransport System was effective for its intended use in achieving topical dermal anaesthesia.

The variables chosen to assess the efficacy of the Electrotransport System in achieving dermal anaesthesia were venipuncture, IV cannulation, incisional or excisional procedures for the removal of superficial skin lesions, and laser treatment for the removal of superficial skin lesions. Both controlled and uncontrolled studies were included in the overall investigational plan.

### Venipuncture and IV Cannulation Studies

Studies show that the Electrotransport System is effective in achieving local dermal anaesthesia for venipuncture, IV cannulation and the laser treatment of superficial skin lesions.

Analyses of the demographic and baseline characteristics are based on data from all subjects enrolled in the studies and randomized to the study treatments. Efficacy analyses are based on the data from all subjects who were administered at least one of the treatments and had efficacy evaluations performed.

Studies 6 and 7

In another set of studies, a total of 548 subjects were enrolled and randomized to the study treatments (276 in Study 6 and 272 in Study 7). A total of 526 subjects were evaluated for efficacy.

Studies 6 and 7 were well-controlled studies conducted in support of the indication of dermal anaesthesia for venipuncture or intravenous (IV) cannulation. Both of these studies were randomized, double-blind, parallel-group, placebo-controlled, prospective, multicenter studies. The first study (Study 6) tested the Electrotransport System in adults (≧18 years of age); while the second study (Study 7) evaluated the system in children 5 to 17 years of age. Both studies compared the performance of the Electrotransport System (administering lidocaine 10% and epinephrine 0.1%) with placebo (an iontophoretic drug delivery system administering buffered saline and epinephrine 0.1%) and evaluated the delivery of dermal anaesthesia in preparation for venipuncture or IV cannulation.

The primary objectives of these studies were to demonstrate the safety and efficacy of the Electrotransport System compared with the placebo system when used for local dermal anaesthesia on intact skin. The results demonstrated that both adult subjects and children ages 5 to 17 years treated with the Electrotransport System reported significantly less pain associated with venipuncture or IV cannulation compared with subjects treated with the placebo system. There were no notable differences in the pain upon venipuncture or IV cannulation among the different age categories in the pediatric subjects treated with the Electrotransport System. In older subjects (12-17 years of age), VAS scores were also used to analyze the pain reported after venipuncture or IV cannulation, allowing for a comparison between children and adults. The results of the VAS scores indicated that the pain perceived by subjects treated with placebo was similar between adult subjects (mean VAS score of 2.53) and pediatric subjects (mean VAS score of 2.58). The mean VAS score for subjects receiving treatment with the Electrotransport System was 0.77 for adult subjects and 1.50 for pediatric subjects.

Study 8

Study 8 was conducted with 61 subjects. All subjects received 1 of 3 treatment combinations consisting of the Platform IIA, Platform IIA placebo, Platform I, and Platform I placebo. The placebo treatments for the respective patches consisted of patch application without current. Of the 61 subjects enrolled in the study, 44 (72.1%) received their assigned treatments and were evaluated for efficacy. A total of 44 (72.1%) subjects completed the study. The age of the subjects ranged from 18 to 57 years.

Anaesthesia (VAS scores), Sensation Associated with Iontophoresis (SAI), and Patch Removal Pain (PRP) were analyzed using the Generalized Linear Model (GLM) procedure to detect the significance of factors (treatment, subject, type of patch, order of treatment administration, and site [hand/antecubital] of administration).

The Sensation Associated with Iontophoresis (SAI) is a scale used to test the sensation associated with iontophoretic delivery. One scale is the VAS scale described above, which is a horizontal liner scale from 0 to 10 where the lowest value represents the least pain or no pain and the highest value represents the most pain, and the other a hedonic scale that measures sensation, which may be pleasurable or painful. The hedonic scale is signed value ±10 cm, as follows:

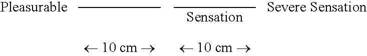

The p-values were based on the raw data and on active-placebo and Platform I-Platform IIA mean differences. The quality of iontophoresis (Hedonic VAS response) was analyzed using the GLM procedure based on normality assumptions. The Patch Removal Pain (PRP) is pain associated with removal of patch after treatment.

For the Platform I, mean VAS scores at the antecubital site were significantly lower for the Electrotransport System than for the placebo (no current) system (1.077 versus 2.780; p=0.0001). Similarly, significant differences were seen between active and placebo (no current) treatments on the hands, where the active treatment was always superior regardless of which of Platforms I or II was used. Comparison of anaesthesia by patch system showed that active treatment with the Platform I provided greater anaesthesia than the Platform IIA; however, this difference was not statistically significant (mean VAS scores of 1.440 versus 2.186; p=0.7101).

Study 8 demonstrated that the Platform I integrated patch provided numerically greater anaesthesia, less sensation of iontophoresis, and better quality of iontophoresis compared with the previously tested Platform IIA integrated patch; however, both were significantly better than the placebo. Placebo treatment (no current) was significantly less effective as an analgesic than active treatment, regardless of the patch system.

Study 9

Study 9 was a double-blind, randomized, placebo-controlled, parallel-group study involving 48 subjects who were evaluated for efficacy. The primary efficacy variable of anaesthesia was assessed immediately after challenge by venipuncture or IV cannulation. Subjects rated their pain intensity from “no pain” to “very severe pain” on a 10-cm visual analogue scale (VAS).

Based on VAS scores recorded immediately after venipuncture or IV cannulation, anaesthesia was significantly (p=0.0001) greater following the active treatment compared with the placebo treatment (combined mean scores of 1.13 versus 3.60, respectively) regardless of the treatment site (antecubital or dorsum of the hand) or challenge type (venipuncture or IV cannulation). Mean VAS scores for the antecubital site and dorsum of the hand, respectively, were 0.66 versus 3.23 and 1.60 versus 3.97 for the active and placebo treatments, respectively. An evaluation by treatment site demonstrated significantly greater pain (p=0.0042) was experienced on the hand dorsum following IV cannulation challenge than at the antecubital site following venipuncture challenge regardless of the treatment type.

The results of this study demonstrated that the Platform I Electrotransport System provided adequate anaesthesia at the antecubital site and on the hand dorsum.

Study 10

Study 10 was conducted with 49 subjects. A total of 24 subjects received treatment with the Platform I Electrotransport System plus current and 25 subjects received treatment with the Platform I without current (placebo). There were 22 males and 27 females ranging in age from 5 to 18 years stratified into three groups (Age Group 1=5 to 7 years (26.5%), Age Group 2=8 to 11 years (34.7%), and Age Group 3=12 to 18 years (38.8%)). Forty-nine (100.0%) subjects completed the study, and 48 (98.0%) subjects were evaluated for efficacy.

Efficacy was assessed by measuring the level of pain subjects experienced during venipuncture after treatment with the Platform I Electrotransport System or placebo. The primary efficacy endpoints were the NFIS measurement used by subjects of all ages and the 10-cm VAS used by subjects of 12 to 18 years of age at the following time points: prior to application of the Platform I patch; immediately after removal of the patch but prior to the blood draw; and after the collection of blood. The secondary endpoints consisted of the CHEOPS Behavioral Assessment and the Overall Experience Questionnaire that were completed after blood was drawn. CHEOPS is the Children's Hospital of Eastern Ontario Pain Scale, a numerical test developed at the Children's Hospital of Eastern Ontario, which is scored by observation by the investigator of the subject for the determination of the level of pain experienced by the subject, particularly children. See, McGrath P J, et al. Adv Pain Res Ther 1985; 9:395-402.

Overall, the mean level of pain experienced during venipuncture was significantly less for subjects treated with the active Platform I than for subjects treated with the placebo patch (2.83 versus 4.32, p=0.016). This effect was observed among the three age groups.

Overall, mean total CHEOPS scores were low, indicating that less pain was experienced, and there were no notable differences between treatment groups or among age groups in the levels of distress displayed by the subjects. However, subjects in Age Group 3 had lower total mean CHEOPS scores than did subjects in Age Group 1. The scores for subjects in Age Group 2 were intermediate.

More subjects in the Electrotransport System group than in the placebo group evaluated the blood collection experience with the patch system as better than previous venipuncture experiences. Similarly, parents/guardians evaluated their child's venipuncture experience as better with the patch system compared with previous experiences. Phlebotomists and nurses generally rated the venipuncture experiences with the patch system as comparable to other blood drawing experiences. The results for each age group were similar to the overall analysis.

The results demonstrated that children treated with the Platform I embodiment of the Electrotransport System experienced significantly less pain during the venipuncture procedure than did subjects treated with the placebo patch, and this effect was observed for all age groups. With respect to the pain experienced during patch removal, there were no significant differences between the treatment groups. Based on the CHEOPS Behavioral Assessment, there were no notable differences between treatment groups in the levels of distress displayed by the children. Overall and across all age groups, the majority of children and their parents/guardians evaluated the venipuncture experience with the Electrotransport System as better than their previous blood draws.

Other studies were also conducted that evaluated the efficacy of the Electrotransport System for other dermal procedures, including the laser treatment of superficial skin lesions and the incisional or excisional removal of superficial skin lesions. The average pain threshold depth and sensory penetration depth were statistically significantly greater for the subjects treated with the active Electrotransport System than for the subjects treated with the placebo. These studies demonstrated the efficacy of the Electrotransport System compared with placebo in achieving local dermal anaesthesia on intact skin in both adults and children for venipuncture/IV cannulation, the treatment of superficial lesions by laser, and the incisional or excisional removal of superficial skin lesions.

### Incision/Excision Studies

Procedures involving incisions and excisions include, without limitation, removal of skin lesions, biopsies, circumcisions, subcutaneous implantation of replacement pacemakers, removal of scar tissue and skin harvesting. Skin lesions may be removed by cutting with a sharp blade, such as a scalpel or a razor, or by scraping or shaving a raised skin lesion, for example with a razor or a looped Curette. Also included are dermabrasion and skin peeling procedures that involve scraping the top dermal layer with razors or a looped Curettes.

The electrically assisted delivery of a local anaesthetic to the region targeted for the procedure is indicated for the removal of skin lesions, such as hyperkeratotic lesions, actinic keratosis, sebhorrheic keratosis, angioma, hemangioma, basal cell epithelioma, squamous cell carcinoma, dermatofibroma, Clarks nevus, cysts, moles, skin tags, skin nodules and warts. High velocity particle ablation, dermabrasion and skin peeling procedures may also benefit from use of electrically assisted delivery of a local anaesthetic.

The following Table I provides examples of lesions slated for surgical removal that can benefit from the local dermal anaesthetic offered by the Electrotransport device described herein. The chart includes a list of specific lesions and a brief explanation thereof in alphabetical order, the number of patients having a lesion of that type removed, the number of patients who needed supplementary anaesthesia, the number of patients who complained of pain greater than 3, using the pain assessment scale described above and the number of patients whose assessment using the Visual Analogue Scale was greater than >4 cm. Of the 88 patients studied, 10, or 21% of patients required supplemental anaesthetic to continue to the completion of the procedure. The supplemental anaesthetic chosen in these cases was a lidocaine injection. It should be noted that even if a lidocaine injection were the primary means of local anaesthetic, secondary injections are commonly given as needed.

TABLE I

| | | | | Pain assess- | VAS |
|---|---|---|---|---|---|
| Indication | Definition | Number | Supplementary | ment (OCAS) >3 | score >4 cm |
| Actinic | A warty lesion, often pre- | 1 | 0 | 0 | 0 |
| Keratosis | malignant, occurring on the sun- | | | | |
| | exposed skin of the face or hands | | | | |
| Angioma | A tumor composed chiefly of | 1 | 0 | 0 | 0 |
| | lymph and blood vessels | | | | |
| Basal Cell | A slow-growing malignant but | 5 | 0 | 0 | 0 |
| Epithelioma | usually non-metastasizing | | | | |
| | skin cancer | | | | |
| Clarks Nevus | Birthmark | 1 | 0 | 0 | 0 |
| Cherry Angioma | | 1 | 0 | 0 | 0 |
| Cyst | A small capsule like sac that | 1 | 1 | 1 | 1 |
| | encloses certain organisms in | | | | |
| | their dormant or larval stage | | | | |
| Dermatofibroma | A benign skin nodule | 2 | 2 | 2 | 2 |
| | consisting mostly of | | | | |
| | fibrous tissue | | | | |
| Hemangioma | A benign skin lesion | 1 | 0 | 0 | 0 |
| | consisting of dense | | | | |
| Hyperkeratotic | Hypertrophy of the cornea or | 2 | 0 | 1 | 1 |
| | the horny layer of the skin | | | | |
| Moles | A small congenital growth | 28 | 2 | 1 | 2 |
| | on the human skin | | | | |
| Scar Revision | Scar | 1 | 0 | 0 | 0 |
| Sebaceous Cyst | A harmless cyst, especially | 1 | 1 | 1 | 0 |
| | on the scalp or face | | | | |
| Sebhorrheic | A superficial, benign, | 19 | 3 | 2 | 3 |
| Keratosis | verrucose lesion consisting | | | | |
| | of proliferating epidermal | | | | |
| | cells enclosing horn cysts | | | | |
| Skin Tags | An outgrowth of epidermal | 19 | 0 | 0 | 0 |
| | and dermal fibrovascular | | | | |
| | tissue | | | | |
| Squamous Cell | | 2 | 0 | 0 | 0 |
| Carcinoma | | | | | |
| Wart | A hard rough lump | 3 | 1 | 1 | 1 |
| | growing on the skin | | | | |
| | TOTAL | 88 | 10 | 9 | 10 |
| | | 100% | ≈11.5% | ≈9.8 | ≈11.5% |

Study 11

Study 11 was an uncontrolled study that evaluated the efficacy of the Electrotransport System in subjects who were undergoing incisional or excisional procedures for the removal of superficial skin lesions. A total of 88 subjects in this study, mostly female ranging in age from 18-82, were evaluated for efficacy. Study 11 was an open-label, non-randomized, prospective study involving subjects who were undergoing incisional or excisional procedures for the removal of superficial skin lesions. Sufficiency of anaesthesia was evaluated using a 7-point Ordered Category Anaesthesia Scale (OCAS) and a Visual Analogue Scale (VAS).

The Ordered Category Anaesthesia Scale is a scale based on the patient's level of discomfort felt because of the laser according to the following chart

| | |
|---|---|
| INTOLERABLE PAIN: Severe Pain | 6 |
| MODERATE PAIN: Interferes with most activities | 5 |
| MILD PAIN: Might interfere with daily activities to a small degree | 4 |
| MILD DISCOMFORT: Would not interfere with daily activities | 3 |
| NOTICEABLE SENSATION: Mild to no discomfort | 2 |
| POSSIBLY SOME SENSATION: Possible sense of pressure or touch | 1 |
| only | |
| NO SENSATION: Unable to feel contact to region | 0 |

As stated above, the Visual Analogue Scale (VAS) is a horizontal liner scale where the lowest value represents the least pain or no pain and the highest value represents the most pain.

The results of Study 11 demonstrated that the Electrotransport System was able to provide most subjects with sufficient dermal anaesthesia during the surgical procedures. Few subjects treated with the Electrotransport System required supplemental anaesthesia in order to complete the incisional or excisional procedures. Anaesthesia and pain assessments demonstrated that most of the subjects treated with the Electrotransport System experienced no or little pain during the surgical procedures.

To support the primary objective of the study, all subjects documented the sufficiency of anaesthesia immediately after the completion of the incisional or excisional procedure by completing the 7-point ordered category anaesthesia scale (OCAS). Intradermal injection of local anaesthesia was permitted if, in the opinion of the Investigator or at the request of the subject, additional anaesthesia was required to complete the procedure. The need for such supplemental anaesthesia was recorded. Subjects also recorded the level of pain they experienced by using a 10-cm VAS; this score was considered a secondary efficacy endpoint.

### Study of Removal of Dermal Lesions by Laser

### Efficacy of the Electrotransport Iontophoretic Lidocaine Drug Delivery System for Anaesthesia Prior to the Treatment of Dermal Lesions by Laser

Additional controlled studies were conducted to further profile the Electrotransport System and to broaden the types of procedures for which the Electrotransport System would be indicated. They provide data for the indications of dermal anaesthesia for the laser treatment of superficial skin lesions. A total of 66 subjects were evaluated for efficacy in these studies.

Open clinical studies were conducted using the Electrotransport System for procedures using medical lasers to remove dermal lesions. A laser produces a collimated beam of energy at a given wavelength. These medical devices are operated at selected power and can run continuously or intermittently. Other controls concern treatment area covered, which can range from a pinpoint to a wide area. The surgeon or dermatologist chooses the laser and its settings depending upon the tissue to be removed and the area and depth of removal needed. Non-limiting examples of various lasers used in the open clinical studies, chosen by the surgeon or dermatologist skilled in the art are presented in Table II below:

TABLE II

| Laser | Medium | Tunable | Pulsed |
|---|---|---|---|
| CO₂ | CO₂ | N | Y |
| HGS Kr | Kr (HgS | N | Y |
| | polarizing | | |
| | prism) | | |
| VascLight ™ | Nd:YAG | Y | Y |
| | (cooled) | | |
| VersaPulse ® | Ho & Nd:YAG | N | Y |
| erbium:YAG | Nd:YAG | N | Y |

The objective of laser treatment is to remove abnormal tissue or clusters of abnormal cells by delivering focused energy at a frequency that will be generally selectively absorbed by the abnormal tissue or cells. Heat is generated and a certain amount of contiguous normal tissue is heated and possibly damaged. The heat and consequent damage causes pain during the treatment and a topical anaesthetic, such as EMLA® cream has heretofore frequently been used before the procedure. These topical anaesthetics can take up to 90 minutes to take effect and leave a residue that has to be removed or may burn off creating additional vapor along with the tissue being ablated. The use of the integrated lidocaine epinephrine Electrotransport System described herein rapidly anesthetizes the site to be treated and leaves no residue.

Study 12

This was a randomized, double-blind, parallel-group, placebo-controlled, prospective study of the Electrotransport System. Study 12 was conducted with 16 male and 0.51 female subjects ranging in age from 9 to 79 (34 Platform I Electrotransport System and 33 placebo system) scheduled to undergo laser treatment of superficial skin lesions such as port wine stains, telangiectasias, lipomas, keloid scars and tattoo removals. Of the 67 subjects enrolled in the study, 66 subjects (98.5%) completed the study and were evaluated for efficacy (34 Electrotransport System and 32 placebo system).

Subjects were treated with a single application of the Electrotransport System (100 mg of lidocaine HCl and 1.05 mg of epinephrine delivered with a total charge of 17 mA·min) or placebo (1.05 mg of epinephrine and saline) administered over 10 minutes. Approximately 20 minutes after the treatment was completed, subjects underwent the scheduled procedure. The application site was evaluated for erythema and edema using the Draize scale at 10 minutes and 24±4 hours following treatment. All subjects were monitored for changes in vital signs and adverse events.

All subjects evaluated the level of pain they experienced from the laser procedure using a 10-cm visual analogue scale (VAS) (primary efficacy variable). They also completed a 7-point ordered category anaesthesia scale (OCAS) to describe the sufficiency of dermal anaesthesia. In addition, the physician performing the procedure completed a VAS evaluation of his/her perception of the subject's pain during the procedure and noted the percent of the original treatment plan that was completed at the time any additional anaesthesia was required.

In Study 12, both mean and weighted mean VAS scores were lower in the Platform I Electrotransport System treatment group (1.57; 2.042 weighted) than in the placebo treatment group (3.72; 4.548 weighted), although the differences were not statistically significant (p=0.380 for the weighted VAS). Although, overall, children tended to report higher scores than did adults, both mean and weighted mean VAS scores were lower for children in the Electrotransport System treatment group (3.13; 4.286 weighted) compared with children in the placebo treatment group (4.87; 7.000 weighted).

Both the mean and weighted mean VAS scores were lower in the Electrotransport System treatment group (1.89; 2.365 weighted) than in the placebo treatment group (4.75; 5.873 weighted), although the differences were not statistically significant (p=0.255 for the weighted VAS). Although, overall, the physician tended to report higher scores for children versus adults, both the mean and weighted mean VAS scores were lower for children in the Electrotransport System treatment group (3.50; 4.657 weighted) compared with children in the placebo treatment group (7.03; 8.950 weighted).

Overall (collapsing across age groups), most subjects in the Electrotransport System treatment group (31; 91.2%) reported OCAS scores of 0 to 3 (no sensation to mild discomfort, respectively). In the placebo treatment group, almost half of the subjects (15; 46.9%) reported OCAS scores of 4 to 6 (mild pain to intolerable pain, respectively). The difference between the 2 treatment groups in the distribution of OCAS scores was statistically significant (p<0.001).

The number of subjects who received supplemental anaesthesia was low (9 subjects overall; 13.6%) and the majority of these subjects (7 of 9; 77.8%) were in the placebo group. It should be noted that even if an anaesthetic injection, such as lidocaine injection, were the primary means of local anaesthetic delivery, secondary injections are given as needed in procedures such as those cited. Typically, the patient requests more local anaesthetic or the physician determines that more is needed.

The Platform I Electrotransport System was demonstrated to be an effective method of achieving local dermal anaesthesia on intact skin prior to the treatment of dermal lesions by laser. Anaesthesia and pain assessments revealed that most subjects treated with the Electrotransport System experienced no or little pain during the surgical procedures compared with the placebo system and that this effect was consistent in both the adult and the pediatric populations. A lower percentage of subjects treated with the Electrotransport System required supplemental anaesthesia in order to complete the laser treatment compared with subjects treated with the placebo system.

Additional clinical studies were conducted with the Electrotransport System: 13, 14, 15 and 16. These studies with earlier prototypes of the Electrotransport System were preliminary in nature; therefore, only brief summaries of the findings will be presented. A total of 364 subjects were evaluated for efficacy in these studies.

### Variation of Charge Densities, Epinephrine Levels

Study 13

Study 13 was a randomized, subject-blinded, evaluator-blinded (to the extent possible), placebo-controlled study conducted with 12 subjects to assess the skin effects, sensations, and tolerability produced by an iontophoretic lidocaine delivery system over a range of charge densities (2.5, 3.4, and 4.2 mA·min/cm²) and epinephrine levels (0.001%, 0.01%, 0.10%, and 0.30%) while maintaining an adequate level of anaesthesia. At medium and high charge densities (3.4 and 4.2 mA·min/cm², respectively), all of the active treatment patches were effective and provided good initial anaesthesia, including those without epinephrine.

Study 14

Study 14 was a randomized, single-blind study conducted with 48 subjects to determine a level of electrical charge density and epinephrine for a 10% lidocaine patch that would achieve anaesthesia within 10 minutes, assess sensation associated with iontophoresis (SAI) under various experimental conditions, assess the degree of anaesthesia with a visual analogue scale (VAS), assess the duration of anaesthesia via sensitivity to von Frey filaments, and determine whether the targeted area under the patch covered the vein sufficiently. The treatment charge densities of 0 (placebo) and 2.5 mA·min/cm² resulted in sensation intensity scores that were less intense than the scores for the treatment with a charge density of 4.2 mA·min/cm². The Sensation Intensity Scale is a vertical scale from 1 to 10, which ranges from 1 to 10, where 1 indicates no sensation and 10 indicates intense sensation.

The data for Hedonic scores indicated that the 2 higher charge densities (3.4 mA·min/cm² and 4.2 mA·min/cm²) tended to result in slightly more unpleasant feelings, while the lower charge density (2.5 mA·min/cm²) and the placebo treatment tended to be associated with slightly more pleasant feelings. Intravenous cannulation pain was highest for the placebo treatment and significantly lower if any of the active treatment charge densities was applied.

Study 15—Comparison Between Platform IIA and a Prior Art (EMLA®) Device

Study 15 demonstrates that delivery of the drug formulation worked better by the iontophoresis than by topical application. This was a randomized, single-blind study conducted with 63 subjects to compare the degree and duration of anaesthesia of a 10% lidocaine patch containing 0.1% epinephrine delivered at 3.4 mA·min/cm² for 10 minutes with the analgesic effects of EMLA®, a topical medication manufactured by AstraZeneca, L.P., applied for 60 minutes and placebo with no current, to assess the effects of a patch containing phenylephrine 1.0% or NaCl 10 mM, to assess sensation and anaesthesia at a low peak current profile, and to assess the sensation associated with smaller cathode patches and/or lower cathode NaCl molarity. The standard patch administered via iontophoresis over 10 minutes provided better anaesthesia than EMLA® administered over 60 minutes for a 20-gauge catheter IV cannulation. The performance of the patch remained substantially the same with modification such as low peak power, added NaCl, or substitution of epinephrine with phenylephrine.

Study 16

Study 16 was a randomized, double-blind study conducted with 85 subjects (80 efficacy-evaluable) to compare the degree and duration of anaesthesia produced by iontophoresis using an integrated patch (Platform IIA) under various treatment conditions: lidocaine concentration—patches containing lidocaine 10% or 5%+epinephrine; effect of epinephrine—patches containing lidocaine 10% with and without epinephrine; duration of iontophoresis—current applied for 5 or 10 minutes; and timing of—IV cannulation —IV cannulation immediately following patch removal or 30 minutes later. Epinephrine significantly enhanced the anaesthetic effect of lidocaine. The degree of anaesthesia was significantly better when IV cannulation was delayed 30 minutes after patch removal. The sensation of iontophoresis was greater when the current was applied for 10 minutes versus 5 minutes.

Study 17—Substitution of Phenylephrine for Epinephrine

The primary objective of Study 17 was to characterize the dermal effects and iontophoretic sensation of an Electrotransport System utilizing varying levels of phenylephrine, rather than epinephrine, over a range of charge densities. The secondary objective of this study was to verify that the chosen levels of phenylephrine and current do achieve clinical analgesia.

For each subject, the Platform III patches (1 anode and 1 cathode patch) was applied at 8 different sites on the volar surface of the forearm (4 different sites on each forearm) Patches containing the following patch combinations were applied for each subject (one to each treatment site; except for the 100 mg lidocaine HCl with 1 mg phenylephrine, which was applied at 2 different sites using 2 different charge densities):

100 mg lidocaine HCl with no phenylephrine

100 mg lidocaine HCl with 0.1 mg phenylephrine

100 mg lidocaine HCl with 1 mg phenylephrine

100 mg lidocaine HCl with 5 mg phenylephrine

100 mg lidocaine HCl with 10 mg phenylephrine

100 mg lidocaine HCl with 3 mg epinephrine (positive control patch)

100 mg lidocaine HCl with no phenylephrine (placebo patch)

Ascending doses of phenylephrine (0.1 mg, 1 mg, 5 mg, and 10 mg) were selected to provide ratios of phenylephrine:lidocaine HCl. The combination of 3.0 mg epinephrine and 100 mg lidocaine HCl was previously shown to be effective and is used as a control in this study.

The areas to be patched were wiped with 70% isopropyl alcohol and allowed to dry for 15 minutes. During this acclimation period, subjects were instructed on the proper use of a Visual Analog Scale (VAS), and a preliminary VAS reading was recorded as a control. The anode patch was applied first, and the cathode patch was applied adjacent to the anode patch.

With the exception of the placebo patch, all patches were activated with charge density levels of 2.55, 3.4, or 4.25 mA·min/cm². Current was not applied to the placebo patch. The positive control patch was delivered with a current level of 4.25 mA·min/cm² for all subjects. The patch containing 100 mg lidocaine HCl with 1.0 mg phenylephrine was delivered using 2 different charge densities per subject. Each of the remaining 4 patch combinations was delivered using a different current level for each subject. For each subject, patches were activated sequentially according to the randomization schedule, with current delivered for 10 minutes.

The iontophoretic controller provided a constant current and variable voltage source of direct current along with a data acquisition system (Keithley K 575 Data Acquisition System [DAS]) for capturing current and voltage measurements during the procedure. A tourniquet was applied to the area above each patch site for 1 minute after completion of iontophoresis to assess for bruising.

Immediately upon the completion of the current activation period, the subject was asked to describe the sensation experienced during iontophoresis. Iontophoretic sensation was measured using the Sensation Associated with Iontophoresis (SAI) VAS and the Hedonic VAS. The patch was removed and the area under the patch was evaluated for dermal effects using Draize scoring (Draize 1990). The skin was re-examined 1 and 24 hours after patch removal, and at 24-hour intervals if skin reactions developed or persisted.

The von Frey touch detection technique was used to assess the degree of analgesia at baseline (before patch application), immediately after the patch was removed, and at 20 minutes after patch removal. At each time period, the evaluator applied 5 serial non-invasive touches using monofilament fibers ranging from 1.65 to 6.65 gauge. The number of touches detected and the number of false-positive response (i.e., touches detected when no filament was applied) were recorded at each time point.

Iontophoretic sensation was measured using 2 visual analog scales, the sensation associated with iontophoresis (SAI) scale and the Hedonic scale.

The SAI scale is a 21-point vertical VAS used to measure the intensity of sensation felt from iontophoresis from “no pain sensation” to “extremely intense” with “0” representing no sensation and “20” representing the greatest intensity of sensation.

The Hedonic scale was used to measure unpleasantness or pleasantness of iontophoretic sensation. Subjects were asked to answer the question “How pleasant/unpleasant did this feel?” by recording a mark on the 21-point horizontal Hedonic VAS. Sensation was characterized as neutral (no sensation, 0) or slightly, mildly, moderately, very, or extremely unpleasant (U1 to U10) or pleasant (P1 to P10).

At each time period, the gauge of the von Frey hair where the subject was first able to detect 3 of the 5 touches was noted and the differences or deltas in detectable gauge size were examined as follows:
The change in responses from baseline (before patch application) to immediately after patch removal (Delta 1);
The change in responses from baseline (before patch application) to 20 minutes after patch removal (Delta 2);
The change in response immediately after patch removal to 20 minutes after patch removal (Delta 3).

Changes in von Frey responses (Delta 1, 2, and 3) were examined by charge densities.

When von Frey responses immediately after patch removal were compared with those at baseline (Delta 1), there was a slight improvement from baseline in the degree of analgesia with increased charge densities, with the greatest change observed between the two lowest charge densities (2.55-mA·min/cm² and 3.4-mA·min/cm²). All patches containing 100 mg lidocaine HCl and phenylephrine with current were superior to the placebo patch. With the exception of the patch containing 100 mg lidocaine HCl with no phenylephrine delivered with current, all treatments with current provided superior degrees of analgesia compared with the placebo patch (100 mg lidocaine HCl with no current).

When von Frey responses assessed 20 minutes after patch removal were compared with those at baseline (Delta 2), responses for patches using the 2 lowest charge densities (2.55- and 3.4-mA·min/cm²) were numerically better than those for patches using the highest current level (4.25-mA·min/cm²) and for the placebo patch. With the exception of the patch containing 100 mg lidocaine HCl with no phenylephrine with current and the patch containing 100 mg lidocaine HCl with 10 mg phenylephrine with current, all active treatment patches provided better degrees of analgesia than the placebo patch.

When von Frey responses assessed 20 minutes after patch removal were compared those assessed immediately after patch removal (Delta 3), the results for the different phenylephrine dose levels were mixed. A decrease in the degree of analgesia with the 4.25-mA·min/cm² current level was noted at the assessment performed 20 minutes after patch removal.

A summary of the SAI VAS scores is provided in Table III. In this study, the greatest level of sensation felt (13=slightly intense) was associated with patches containing 1 mg and 10 mg phenylephrine. For all other treatments, the level of sensation ranged from no sensation to moderate sensation.

TABLE III
Sensation Associated With Iontophoresis (SAI) VAS Scores

| Treatment¹ | SAI VAS Scores² (Range) |
|---|---|
| No phenylephrine³ | 1 (faint) to 9 (mild) |
| 0.1 mg phenylephrine³ | 4 (very weak) to 11 (moderate) |
| 1 mg phenylephrine³ | 8 (mild) to 13 (slightly intense) |
| 5 mg phenylephrine³ | 10 (mild) to 11 (moderate) |
| 10 mg phenylephrine³ | 5 (weak) to 13 (slightly intense) |
| Placebo patch⁴ | 0 (no sensation) to 10 (mild) |
| 3 mg epinephrine⁵ | 4 (very weak) to 11 (moderate) |

¹All treatments also contained 100 mg lidocaine HCl.
²21-point visual analog scale, where 0 = no sensation and 20 = the highest level of intensity.
³All charge densities.
⁴100 mg lidocaine HCl with no current.
⁵Delivered with a charge density of 4.25 mA · min/cm².

A summary of the Hedonic VAS scores is provided in Table IV. The sensation felt with patches containing no phenylephrine (100 mg lidocaine HCl alone) and the placebo patch were characterized as producing neutral to extremely pleasant sensations. For all other treatments, sensation was characterized as slightly unpleasant to slightly pleasant.

TABLE IV
Hedonic VAS Scores

| Treatment¹ | Hedonic VAS Scores² (Range) |
|---|---|
| No phenylephrine³ | neutral to moderately pleasant |
| 0.1 mg phenylephrine³ | slightly unpleasant to slightly pleasant |
| 1 mg phenylephrine³ | moderately unpleasant to neutral |
| 5 mg phenylephrine³ | slightly unpleasant to neutral |
| 10 mg phenylephrine³ | moderately unpleasant to slightly pleasant |
| Placebo patch⁴ | neutral to extremely pleasant |
| 3 mg epinephrine⁵ | mildly unpleasant to slightly unpleasant |

¹All treatments also contained 100 mg lidocaine HCl.
²21-point visual analog scale, sensation was characterized as neutral (no sensation) or slightly, mildly, moderately, very, or extremely pleasant or unpleasant.
³All charge densities.
⁴100 mg lidocaine HCl with no current.
⁵Delivered with a charged density of 4.25 mA · min/cm².

### Efficacy Conclusions

Different phenylephrine levels, in combination with 100 mg lidocaine HCl, produced analgesia after a 10-minute delivery interval. Except when delivered with a current of 4.25 mA·min/cm², the analgesic effect persisted for at least 20 minutes after patch removal. Sensation associated with iontophoresis was characterized as very weak to slightly intense (scores of 4 to 13) on the SAI VAS scale (0 to 20), and moderately unpleasant to slightly pleasant on the Hedonic VAS scale.

Different phenylephrine levels, in combination with 100 mg lidocaine HCl, produced analgesia after a 10-minute delivery interval. Except when delivered with a current density of 4.25 mA·min/cm², the analgesic effect persisted for at least 20 minutes after patch removal. Sensation associated with iontophoresis was characterized as very weak to slightly intense (scores of 4 to 13) on the SAI VAS scale (0 to 20), and moderately unpleasant to slightly pleasant on the Hedonic VAS scale.

1-19. (canceled)

20. A method of topically anaesthetizing a portion of the skin of a patient prior to a procedure to be preformed on the patient comprising:
administering through a patient's intact skin a drug formulation comprised of an anaesthetic and a vasoconstrictor in an amount sufficient for clinically acceptable depth and duration of dermal anaesthesia by application to the patient's skin, an iontophoresis electrode assembly having an anode assembly, including a pre-loaded hydrogel drug reservoir in electrical contact with a first electrode, said procedure comprising one of venipuncture, IV cannulation, needle aspiration, body piercing, blood donation, electrolysis, tattoo removal, tattoo application, injections, dermabrasion, skin peeling, high velocity particle ablation, pace maker implantation, pace maker replacement, epidural puncture, lumbar puncture, a regional nerve block, skin harvesting, skin incisions, skin biopsies, circumcisions, excisions and the treatment of neuropathic pain.

21. The method of claim 20 wherein the iontophoresis is for the administration of an initial relatively minor dose of a topical anaesthetic prior to the injection of a major dose of anaesthesia.

22. The method of claim 20 wherein the anaesthetic is selected from the group consisting of amide type anaesthetics, ester type anaesthetics, bupivacaine, butanilicaine, carticaine, cinchocaine/dibucaine, clibucaine, ethyl parapiperidino acetylaminobenzoate, etidocaine, lidocaine, mepivicaine, oxethazaine, prilocaine, ropivicaine, tolycaine, trimecaine, vadocaine, amylocaine, cocaine, propanocaine, esters of metaminobenzoic acid, clormecaine, proxymetacaine, esters of paraminobenzoic acid, amethocaine, benzocaine, butacaine, butoxycaine, butyl aminobenzoate, chloroprocaine, oxybuprocaine, parethoxycaine, procaine, propoxycaine, tricaine, bucricaine, dimethisoquin, diperodon, dyclocaine, ethyl chloride, ketocaine, myrtecaine, octacaine, pramoxine and propipocaine.

23. The method of claim 20 wherein the anaesthetic is one of bupivacaine, butacaine, chloroprocaine, cinchocaine, etidocaine, mepivacaine, prilocaine, procaine, ropivacaine and tetracaine.

24. The method of claim 20 wherein the anaesthetic is one of bupivacaine, etidocaine, mepivacaine, ropivicaine and prilocaine.

25. The method of claim 20 wherein the anaesthetic is lidocaine.

26. The method of claim 20 wherein the vasoconstrictor is epinephrine.

27. The method of claim 20 wherein the depth of anaesthesia is at least 2.5 mm.

28. A method of topically anaesthetizing a portion of the skin of a patient prior to a procedure to be preformed on the patient comprising:
administering through a patient's intact skin a drug formulation comprised of an anaesthetic in an amount sufficient for clinically acceptable depth and duration of dermal anaesthesia by application to the patient's skin, an iontophoresis electrode assembly having an anode assembly, including a pre-loaded hydrogel drug reservoir in electrical contact with a first electrode, said procedure comprising the treatment of refractory pain resulting from cancer, diabetic neuropathy, neuropathy brought on by Shingles, and pain from trigeminal and postherpetic neuralgia.

29. A method of producing local anaesthesia in a patient prior to a procedure, comprising:
applying a charge density of at least about 3.4 mA·min/cm² for at least about 5 minutes to an electrically assisted drug delivery system comprising an anode assembly including a reservoir in electrical contact with the patient, wherein the reservoir is loaded with a drug formulation including an anaesthetic and a vasoconstrictor, the electrically assisted drug delivery system producing clinically acceptable depth and duration of dermal anaesthesia at a treated site, wherein the average depth to which all sensation is eliminated on advancing an 18 gauge needle into the treated skin of a forearm of a patient immediately after treatment with the electrode assembly and the drug formulation is greater than 5 mm and the procedure is one of venipuncture, IV cannulation, needle aspiration, body piercing, blood donation, electrolysis, tattoo removal, tattoo application, injections, dermabrasion, skin peeling, high velocity particle ablation, pace maker implantation, pace maker replacement, epidural puncture, lumbar puncture, a regional nerve block, skin harvesting, skin incisions, skin biopsies, circumcisions, excisions and the treatment of neuropathic pain.

30. The method of claim 29 wherein the vasoconstrictor is present in amounts not greater than 0.5% by weight and the neuropathic pain is refractory pain resulting from cancer, diabetic neuropathy, neuropathy brought on by Shingles, and trigeminal and postherpetic neuralgia treatment.

31. The method of claim 29 wherein the injection is for the administration of an initial relatively minor dose of a topical anaesthetic prior to the injection of a major dose of anaesthesia.

32. The method of claim 29 wherein the anaesthetic is selected from the group consisting of amide type anaesthetics, ester type anaesthetics, bupivacaine, butanilicaine, carticaine, cinchocaine/dibucaine, clibucaine, ethyl parapiperidino acetylaminobenzoate, etidocaine, lidocaine, mepivicaine, oxethazaine, prilocaine, ropivicaine, tolycaine, trimecaine, vadocaine, amylocaine, cocaine, propanocaine, esters of metaminobenzoic acid, clormecaine, proxymetacaine, esters of paraminobenzoic acid, amethocaine, benzocaine, butacaine, butoxycaine, butyl aminobenzoate, chloroprocaine, oxybuprocaine, parethoxycaine, procaine, propoxycaine, tricaine, bucricaine, dimethisoquin, diperodon, dyclocaine, ketocaine, myitecaine, octacaine, pramoxine and propipocaine.

33. The method of claim 29, wherein the procedure is one of venipuncture, IV cannulation, needle aspiration, body piercing, blood donation, epidural puncture, lumbar puncture, or a regional nerve block and the average pain threshold on advancing an 18 gauge needle into the treated skin of a patient after treatment with the electrode assembly and the drug formulation does not decrease within the first hour immediately after ending the treatment.

34. The method of claim 20 wherein the anaesthetic is lidocaine and the vasoconstrictor is epinephrine.

35. The method of claim 34 wherein the applied current density and the duration of the delivery of the drug formulation are such that systemic delivery of the anaesthetic and the vasoconstrictor is avoided.

36. The method of claim 34 wherein the duration of the delivery of the drug formulation is about 10 minutes and the electric charge is about 17.7 mAmin.

37. A method of topically anaesthetizing a portion of the skin of a patient prior to an excision or incision of said portion of skin comprising:
administering through a patient's intact skin a drug formulation comprised of an anaesthetic and a vasoconstrictor in an amount sufficient for clinically acceptable depth and duration of dermal anaesthesia by application to a portion of the patient's skin, an iontophoresis electrode assembly having an anode assembly, including a pre-loaded hydrogel drug reservoir in electrical contact with a first electrode; passing current for at least 5 minutes; and, waiting for the portion of skin to be anaesthetized.

38. The method of claim 37 wherein the excision is for the removal of one or more of a cyst, a wart, a mole, scar tissue, skin nodules, skin tags, angiomas, sebhorrheic keratosis, actinic keratosis, and hemangiomas.

39. The method of claim 37 wherein the excision removes one or more of birthmarks and tattoos.

40. The method of claim 37 wherein the anaesthetic is selected from the group consisting of amide type anaesthetics, ester type anaesthetics, bupivacaine, butanilicaine, carticaine, cinchocaine/dibucaine, clibucaine, ethyl parapiperidino acetylaminobenzoate, etidocaine, lidocaine, mepivicaine, oxethazaine, prilocaine, ropivicaine, tolycaine, trimecaine, vadocaine, amylocaine, cocaine, propanocaine, esters of metaminobenzoic acid, clonnecaine, proxymetacaine, esters of paraminobenzoic acid, amethocaine, benzocaine, butacaine, butoxycaine, butyl aminobenzoate, chloroprocaine, oxybuprocaine, parethoxycaine, procaine, propoxycaine, tricaine, bucricaine, dimethisoquin, diperodon, dyclocaine, ketocaine, myrtecaine, octacaine, pramoxine and propipocaine.

41. The method of claim 40 wherein the vasoconstrictor is epinephrine.

42. The method of claim 37 wherein the anaesthetic is lidocaine and the vasoconstrictor is epinephrine.

43. The method of claim 37 wherein the iontophoresis electrode assembly comprises:
a flexible backing;
an electrode layer connected to said flexible backing, said electrode layer having at least a donor electrode and a return electrode;
at least one lead extending from each of said donor electrode and said return electrode to a tab end portion of said assembly, said tab end portion being structured for electrical connection with at least one component of said electrically assisted delivery device;
a donor reservoir positioned in communication with said donor electrode, said donor reservoir including an amount of said composition;
a return reservoir positioned in communication with said return electrode; and, at least one of the following: (a) an insulating dielectric coating positioned adjacent to at least a portion of at least one of said electrodes and said leads, (b) at least one spline formed in said electrode layer, (c) a tab stiffener connected to said tab end portion, (d) a tab slit formed in said tab end portion, (e) a sensor trace positioned on said tab end portion, (f) a release cover having a donor portion structured to cover said donor reservoir and a return portion structured to cover said return reservoir, (g) at least a portion of said flexible backing having a flexural rigidity less than a flexural rigidity of at least a portion of said electrode layer, (h) wherein a shortest distance between a surface area of an assembly including said donor electrode and said donor reservoir and a surface area of an assembly including said return electrode and said return reservoir being sized to provide a substantially uniform path of delivery for said composition through said membrane, (i) wherein a surface area of an assembly including said donor electrode and said donor reservoir is greater than a surface area of an assembly including said return electrode and said return reservoir, (j) wherein a ratio of a surface area of at least one of said reservoirs to a surface area of its corresponding electrode is in the range of about 1.0 to 1.5, (k) wherein a footprint area of said assembly is in the range of about 5 cm².sup.2 to 100 cm², (l) wherein a ratio of a total surface area of said electrodes to a total footprint area of said assembly is in the range of about 0.1 to 0.7, (m) wherein a ratio of a surface area of said donor electrode to a surface area of said return electrode is in the range of about 0.1 to 5.0, (n) wherein a ratio of a thickness of said donor reservoir to a thickness of said return reservoir is in the range of about 0.2 to 3.0, (o) wherein at least one component of said assembly in communication with at least one of said reservoirs has an aqueous absorption capacity less than an aqueous absorption capacity of said reservoir in communication with said component of said assembly, (p) a slit formed in said flexible backing in an area located between said donor electrode and said return electrode, (q) at least one non-adhesive tab extending from said flexible backing, (r) a gap formed between a portion of a layer of transfer adhesive deposited on said electrode layer and a portion of a tab stiffener connected to said tab end portion, (s) at least one tactile sensation aid formed in said tab end portion, (t) at least one indicium formed on at least a portion of said assembly, (u) a minimum width of a portion of a layer of transfer adhesive deposited on said electrode layer adjacent to at least one of said donor electrode and said return electrode is in the range of at least about 0.9 cm, (v) a minimum tab length associated with said tab end portion is in the range of at least about 3.5 cm.

44-47. (canceled)

48. A method of inducing analgesia in skin or tissue, comprising topically administering to a patient in need of such treatment a topically analgesically effective amount of anaesthetic admixed with a vasoconstrictor sufficient for performing one of the procedures selected from the group consisting of venipuncture, IV cannulation, needle aspirations, body piercings, needle injections for blood donations, electrolysis, tattoo removal, tattoo application, injections, dermabrasion, skin peeling, high velocity particle ablation, pace maker implantation, pace maker replacement, epidural puncture, lumbar puncture, regional nerve blocks, skin harvesting, small skin incisions, skin biopsies, circumcisions, excisions and the treatment of neuropathic pain, the administration by means of an integrated electrode assembly structured for use in association with an electrically assisted delivery device for delivery of the composition said assembly comprising: a flexible backing; an electrode layer connected to said flexible backing, said electrode layer having at least a donor electrode and a return electrode; at least one lead extending from each of said donor electrode and said return electrode to a tab end portion of said assembly, said tab end portion being structured for electrical connection with at least one component of said electrically assisted delivery device; a donor reservoir positioned in communication with said donor electrode, said donor reservoir including an amount of the composition; a return reservoir positioned in communication with said return electrode; and, an insulating dielectric coating positioned adjacent to at least a portion of at least one of said electrodes and said leads.

49. The method of claim 48 wherein the neuropathic pain is refractory pain resulting from cancer, diabetic neuropathy, neuropathy brought on by Shingles, and trigeminal and postherpetic neuralgia treatment.

50. The method of claim 48 wherein the injection is for the administration of a dose of anaesthetic.

51. The method of claim 48 wherein the anaesthetic is selected from the group consisting of amide type anaesthetics, ester type anaesthetics, bupivacaine, butanilicaine, carticaine, cinchocaine/dibucaine, clibucaine, ethyl parapiperidino acetylaminobenzoate, etidocaine, lidocaine, mepivicaine, oxethazaine, prilocaine, ropivicaine, tolycaine, trimecaine, vadocaine, amylocaine, cocaine, propanocaine, esters of metaminobenzoic acid, clonnecaine, proxymetacaine, esters of paraminobenzoic acid, amethocaine, benzocaine, butacaine, butoxycaine, butyl aminobenzoate, chloroprocaine, oxybuprocaine, parethoxycaine, procaine, propoxycaine, tricaine, bucricaine, dimetlisoquin, diperodon, dyclocaine, ketocaine, myrtecaine, octacaine, pramoxine and propipocaine.

52. The method of claim 48 wherein the anaesthetic is one of bupivacaine, etidocaine, mepivacaine, ropivicaine and prilocaine.

53. The method of claim 48 wherein the anaesthetic is lidocaine.

54. A method of anaesthetizing a topical section of a patient's skin prior to excision of skin lesions, tumors, birthmarks, cysts, moles, warts, skin nodules, scar revision, skin tags, sebhorrheic keratosis, skin harvesting and dermabrasion by application of a composition including an anaesthetic and a vasoconstrictor by iontophoresis with an integrated electrode assembly structured for use in association with an electrically assisted delivery device for delivery of said composition through a membrane, said assembly comprising: a flexible backing; an electrode layer connected to said flexible backing, said electrode layer having at least a donor electrode and a return electrode; at least one lead extending from each of said donor electrode and said return electrode to a tab end portion of said assembly, said tab end portion being structured for electrical connection with at least one component of said electrically assisted delivery device; a donor reservoir positioned in communication with said donor electrode, said donor reservoir including an amount of the composition; a return reservoir positioned in communication with said return electrode; and, an insulating dielectric coating positioned adjacent to at least a portion of at least one of said electrodes and said leads.
